(19) United States
(12) Patent Application Publication
(10) Pub. No.: US 2025/0049736 A1
(43) Pub. Date: Feb. 13, 2025

(54) SULFONAMIDE DERIVATIVES AND THEIR USE AS SOLUBLE EPOXIDE HYDROLASE INHIBITORS

(71) Applicant: THE UNITED STATES OF AMERICA, AS REPRESENTED BY THE SECRETARY, DEPARTMENT OF HEALTH, Bethesda, MD (US)

(72) Inventors: Malliga R. Iyer, Germantown, MD (US); Biswajit Kundu, Rockville, MD (US)

(21) Appl. No.: 18/718,691

(22) PCT Filed: Dec. 15, 2022
(86) PCT No.: PCT/US2022/052957

§ 371 Date: Jun. 11, 2024

Related U.S. Application Data
(60) Provisional application No. 63/290,409, filed on Dec. 16, 2021.

(51) Int. Cl. A61K 31/155; A61K 45/06; A61P 37/00; C07C 311/59

(52) U.S. Cl. CPC A61K 31/155; A61K 45/06; A61P 37/00; C07C 311/59

(57) ABSTRACT

The present invention relates to compounds of formulae I and II. The compounds are useful for treating hypertension, atherosclerosis, pulmonary diseases, diabetes, pain, fibrosis, addictive disorders, inflammation, and immunological disorders or a condition treatable or preventable by inhibition of soluble epoxide hydrolase. Preferred compounds are N-((adamantan-1-yl)carbamoyl)-benzenesulfonamide derivatives. Exemplary compounds are e.g. •N—(((1r,3s,5R,7S)-3-hydroxyadamantan-1-yl)carbamoyl)-4-methylbenzenesulfonamide (example 14, compound 4a) •4-(tert-butyl)-N—(((1r,3s,5R,7S)-3-hydroxyadamantan-1-yl)carbamoyl)benzenesulfonamide (example 15, compound 4b) •N—(((1r,3s,5R,7S)-3-hydroxyadamantan-1-yl)carbamoyl)-4-(trifluoromethyl)benzenesulfonamide (example 16, compound 4j). The present description discloses the synthesis and characterisation of exemplary compounds as well as pharmacological data thereof.

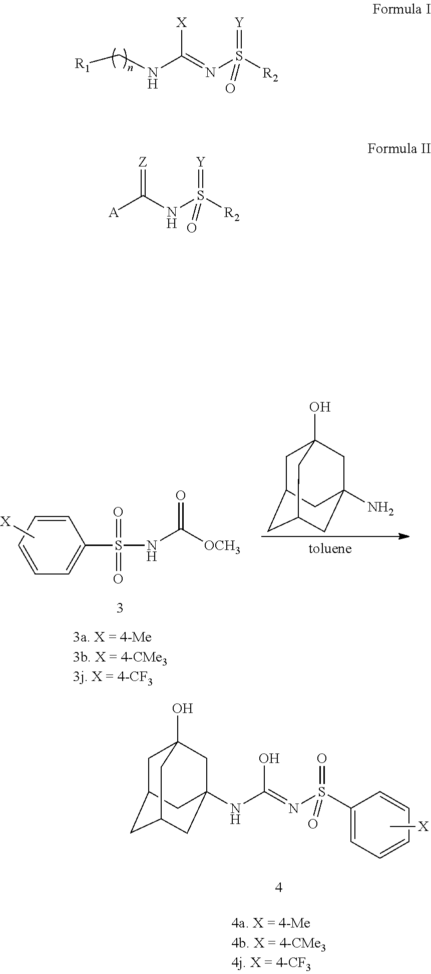

### CROSS REFERENCE TO RELATED APPLICATION

This application claims priority of U.S. Provisional Application No. 63/290,409, filed Dec. 16, 2021, which is hereby incorporated by reference in its entirety.

### STATEMENT OF GOVERNMENT SUPPORT

This invention was made in part with government support from the US Department of Health and Human Services, National Institutes of Health. The Government has certain rights in this invention.

### BACKGROUND OF THE INVENTION

### 1. Field of the Invention

This invention relates generally to epoxide hydrolase inhibiting compounds, and more specifically to soluble epoxide hydrolase inhibiting compounds that offer additive therapeutic efficacy or novel synergisms in treating multi-etiological disease conditions, as well as possible applications as biologically important probes and drugs, imaging-fluorescent probes and labels, and in environmental applications.

### 2. Brief Description of the Art

The enzyme soluble epoxide hydrolase (sEH) has a pivotal role in the metabolism process of bioactive lipid signaling molecules. The substrate-specific hydrolase activity of sEH transforms epoxyeicosatrienoic acids (EETs) to the corresponding dihydroxyeicosatrienoic acids (DHETs) with lesser bioactivity. It has been demonstrated that sEH inhibition leads to elevated levels of EETs, which subsequently exhibit various beneficial biological effects. Hence, sEH inhibition is an important therapeutic strategy for the treatment of a variety of diseases, and mammalian soluble epoxide hydrolase (sEH) represents a promising new target for drug development. Chemical inhibition of sEH in animal models has been demonstrated to have beneficial effects on the treatment of hypertension and vascular inflammation as well as related syndromes.

The X-ray co-crystal structure of human sEH complexed with the sEH inhibitor 4-(3-cyclohexyluriedo) butyric acid (PDB code 1ZD3) revealed the catalytic pocket and the structural features that may contribute to the inhibition of sEH. The epoxide hydrolase catalytic pocket contains two tyrosine residues (Tyr381 and Tyr465) that catalyse the epoxide ring-opening by Asp333. The resulting ester further gets rapidly hydrolyzed into the corresponding DHETs. It has been identified that amide, carbamate, and urea groups fit well in the hydrolase catalytic pocket. Specifically, the carbonyl oxygen of amide or urea can be involved in hydrogen bond formation with Tyr381 and Tyr465, and the NH of ureas or amides may serve as a hydrogen bond donor to Asp333. Various urea and amide derivatives have been developed as reversible, and often tight binding sEH inhibitors. However, although existing sEH inhibitors are relatively potent and specific, low solubility and relatively fast metabolism decrease their therapeutic efficiency. sEH inhibitors have been in clinical trials in the past and are currently in clinical trial as well. However clinically approved agents targeting this pathway are not yet available. Thus there is an unmet need for the development of improved soluble epoxide hydrolase inhibitors.

The present invention is believed to be an answer to that need.

### SUMMARY OF THE INVENTION

In an aspect, the present invention is directed to a compound of Formula I or a pharmaceutically acceptable ester, amide, solvate, salt, prodrug, or metabolite thereof, or a salt of such an ester, amide, prodrug, or metabolite, or a solvate of such an ester, amide, salt, prodrug, or metabolite,

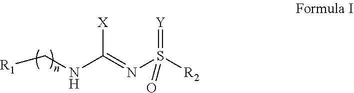

wherein in Formula I:
n is 0 or 1;
R₁ is a cycloalkyl, and the cycloalkyl is unsubstituted or substituted with one or more substituents independently chosen from halogen, hydroxyl, amino, (mono- or di-C₁-C₆alkylamino)C₀-C₄alkyl, C₁-C₆alkyl, C₁-C₆alkoxy, C₁-C₆haloalkyl, and C₁-C₆haloalkoxy;
R₂ is an aryl or heteroaryl, and the aryl and the heteroaryl are unsubstituted or substituted with one or more substituents independently chosen from halogen, hydroxyl, cyano, nitro, amino, (mono- or di-C₁-C₆alkylamino)C₀-C₄alkyl, C₁-C₆alkyl, C₁-C₆alkoxy, C₁-C₆haloalkyl, C₁-C₆haloalkoxy, aryl, —COOR₁₆, —CONR₁₆R₁₆, —SO₂NR₁₆R₁₆, —B(OR₁₆)₃, —P(═O)(OR₁₆)₃, —NHNH₂, and triazolyl, wherein each R₁₆ is independently hydrogen or C₁-C₆ alkyl; or
R₂ is —NR₁₁R₁₂ or —N(CH₂)ₓ wherein R₁₁ and R₁₂ are each independently hydrogen, C₁-C₆alkyl, or cycloalkyl, x is 4 to 6, and any one CH₂ in the —N(CH₂)ₓ is optionally replaced by NR₁₈, O, S, or SO₂, wherein R₁₈ is hydrogen or C₁-C₆ alkyl, and the cycloalkyl and —N(CH₂)ₓ are unsubstituted or substituted with one or more substituents independently chosen from halogen, hydroxyl, amino, (mono- or di-C₁-C₆alkylamino)C₀-C₄alkyl, C₁-C₆alkyl, C₁-C₆alkoxy, C₁-C₆haloalkyl, and C₁-C₆haloalkoxy;
X is —OR, —NHR, —SR(R₁₃)ₘ, —SeR(R₁₃)ₘ, —C(R)₃, —N(CH₂)_{b}SO₂R₁₄, —N(CH₂)_{b}COOR₁₄, —N(CH₂)_{b}COR₁₄, —N(CH₂)_{b}POR₁₄, —N(CH₂)_{b}BR₁₄, or —N═CR₃R₄, wherein each R is independently hydrogen, amino, cyano, or C₁-C₆alkyl with one or more methylene groups optionally replaced by an oxygen, R₁₃ is an oxo group, m is 0, 1, or 2, b is 0 to 6, R₁₄ is hydrogen or C₁-C₆alkyl, and R₃ and R₄ are each independently C₁-C₆alkyl, —SR(R₁₃)ₘ, —SeR(R₁₃)ₘ, —NHR, or R₃ and R₄ are joined to form a ring optionally containing one additional heteroatom chosen from N, O, and S and the ring is unsubstituted or substituted with one or more substituents independently chosen from halogen, hydroxyl, cyano, nitro, amino, (mono- or di-C₁-C₆alkylamino)C₀-C₄alkyl, C₁-C₆alkyl, C₁-C₆alkoxy, C₁-C₆haloalkyl, and C₁-C₆haloalkoxy; and
Y is O or NR₁₀, wherein R₁₀ is hydrogen or C₁-C₆alkyl.

In another aspect, the present invention is directed to a compound of Formula II, Formula II-B or a pharmaceutically acceptable ester, amide, solvate, salt, prodrug, or metabolite thereof, or a salt of such an ester, amide, prodrug, or metabolite, or a solvate of such an ester, amide, salt, prodrug, or metabolite,

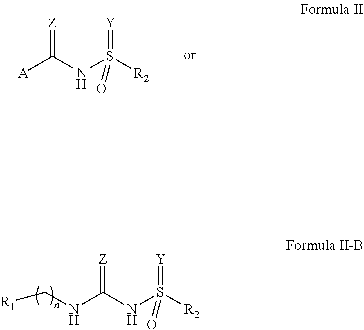

wherein in Formula II and Formula II-B:
A is

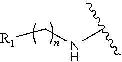

or —NR₂₁R₂₂;
n is 0 or 1;
R₁ is a cycloalkyl or

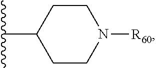

and the cycloalkyl is unsubstituted or substituted with one or more substituents independently chosen from halogen, hydroxyl, amino, (mono- or di-C₁-C₆alkylamino)C₀-C₄alkyl, C₁-C₆alkyl, C₁-C₆alkoxy, C₁-C₆haloalkyl, and C₁-C₆haloalkoxy;
R₆₀ is hydrogen, —C(O)—R₇₀, or —C(O)NHR₇₀, R₇₀ is C₁₋₆alkyl or cycloalkyl;
R₂₁ and R₂₂ are each independently hydrogen, C₁₋₆alkyl, or cycloalkyl, or R₂₁, R₂₂, together with the nitrogen atom in —NR₂₁R₂₂ form a five membered or six membered heterocycloalkyl, which is unsubstituted or substituted with one or more substituents independently chosen from halogen, hydroxyl, amino, (mono- or di-C₁-C₆alkylamino)C₀-C₄alkyl, C₁-C₆alkyl, C₁-C₆alkoxy, C₁-C₆haloalkyl, and C₁-C₆haloalkoxy;
R₂ is an aryl or heteroaryl, and the aryl and the heteroaryl are unsubstituted or substituted with one or more substituents independently chosen from halogen, hydroxyl, cyano, nitro, amino, (mono- or di-C₁-C₆alkylamino)C₀-C₄alkyl, C₁-C₆alkyl, C₁-C₆alkoxy, C₁-C₆haloalkyl, C₁-C₆haloalkoxy, aryl, —COOR₁₆, —CONR₁₆R₁₆, —SO₂NR₁₆R₁₆, —B(OR₁₆)₃, —P(═O)(OR₁₆)₃, —NHNH₂, and triazolyl, wherein each R₁₆ is independently hydrogen or C₁-C₆ alkyl; or
R₂ is —NR₁₁R₁₂ or —N(CH₂)ₓ, wherein R₁₁ and R₁₂ are each independently hydrogen, C₁-C₆alkyl, or cycloalkyl, x is 4 to 6, and any one CH₂ in the —N(CH₂)ₓ is optionally replaced by NR₁₈, O, S, or SO₂, wherein R₁₈ is hydrogen or C₁-C₆ alkyl, and the cycloalkyl and —N(CH₂)ₓ are unsubstituted or substituted with one or more substituents independently chosen from halogen, hydroxyl, amino, (mono- or di-C₁-C₆alkylamino)C₀-C₄alkyl, C₁-C₆alkyl, C₁-C₆alkoxy, C₁-C₆haloalkyl, and C₁-C₆haloalkoxy;
Y is O or NR₁₀, wherein R₁₀ is hydrogen or C₁-C₆alkyl; and
Z is O, NR₁₅ or S, wherein R₁₅ is hydrogen or C₁-C₆alkyl,
with the proviso that R₁ is not adamantyl when a is zero, Z and Y are O, and R₂ is 4-methylphenyl.

In another aspect, the present invention is directed to a pharmaceutical composition that includes a compound of Formula I or Formula II or a pharmaceutically acceptable ester, amide, solvate, salt, prodrug, or metabolite thereof, or a salt of such an ester, amide, prodrug, or metabolite, or a solvate of such an ester, amide, salt, prodrug, or metabolite, together with a pharmaceutically acceptable carrier.

In yet another aspect, the present invention is directed to a method of treating an immunological disorder, or a condition treatable or preventable by inhibition of soluble epoxide hydrolase in a patient that includes providing to a patient in need thereof a compound of Formula I or Formula II or a pharmaceutically acceptable ester, amide, solvate, salt, prodrug, or metabolite thereof, or a salt of such an ester, amide, prodrug, or metabolite, or a solvate of such an ester, amide, salt, prodrug, or metabolite, or a pharmaceutical composition described herein above.

The present invention also utilizes a fragment-assisted dual-target/multi-target approach for improving efficacy of sEH inhibitors. The compounds disclosed here are not only s-EH inhibitors but can also modulate additional synergistic/complementary signaling pathways (either as a pro-drug, metabolite or the whole drug) (for e.g, iNOS, Caspase-1, NLRP3, CB2R, AMPK, GABAA etc) in treating disease conditions.

### BRIEF DESCRIPTION OF THE DRAWING

FIGS. 1-4 show the fluorescence assay results of exemplified compounds;

FIGS. 5A, 5B, 5C, and 5D are graphs of sEH activity (%) versus log concentration (M) of AUDA, compounds 5d, 5e, and 5i respectively;

FIGS. 6A, 6B, 6C, and 6D are graphs of sEH activity (%) versus log concentration (M) of compounds 5f, 5g, 7i, and 8i respectively; and

FIGS. 7A, 7B, 7C, and 7D are graphs of sEH activity (%) versus log concentration (M) of compounds 9i, 11c, 11d, and 13i respectively.

### DETAILED DESCRIPTION OF THE INVENTION

### Terminology

Compounds of the present disclosure are generally described using standard nomenclature.

The terms “a” and “an” do not denote a limitation of quantity, but rather denote the presence of at least one of the referenced item. The term “or” means “and/or.” The open-ended transitional phrase “comprising” encompasses the intermediate transitional phrase “consisting essentially of” and the close-ended phrase “consisting of.” Claims reciting one of these three transitional phrases, or with an alternate transitional phrase such as “containing” or “including” can be written with any other transitional phrase unless clearly precluded by the context or art.

Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable.

All methods described herein can be performed in a suitable order unless otherwise indicated herein or otherwise clearly contradicted by context. The use of any and all examples, or exemplary language (e.g., “such as”), is intended merely to better illustrate the disclosure and does not pose a limitation on its scope unless otherwise claimed. No language in the specification should be construed as indicating any non-claimed element as essential to the practice of the invention as used herein. Unless defined otherwise, technical and scientific terms used herein have the same meaning as is commonly understood by one of skill in the art to which this disclosure belongs.

Formulae I and II include all subformulae such as Formula I-A, Formula II-A, Formula II-B, and Formula II-C.

The compounds described herein include tautomers and polymorphs.

In certain situations, the compounds described herein may contain one or more asymmetric elements such as stereogenic centers, stereogenic axes and the like, e.g. asymmetric carbon atoms, so that the compounds can exist in different stereoisomeric forms. The disclosed compounds include all stereoisomeric forms, including racemates, optically enriched, and optically pure forms. In addition, compounds with carbon-carbon double bonds may occur in Z- and E-forms, with all isomeric forms of the compounds being included in the present disclosure.

In these situations, the single enantiomers, i.e., optically active forms can be obtained by asymmetric synthesis, synthesis from optically pure precursors, or by resolution of the racemates. Resolution of the racemates can also be accomplished, for example, by conventional methods such as crystallization in the presence of a resolving agent, or chromatography, using, for example, a chiral HPLC column.

The disclosure of compounds includes all isotopes of atoms occurring in the present compounds. Isotopes include those atoms having the same atomic number but different mass numbers. By way of general example, and without limitation, isotopes of hydrogen include tritium and deuterium and isotopes of carbon include ¹¹C, ¹³C, and ¹⁴C and isotopes of fluorine including ¹⁹F.

Certain compounds are described herein using a general formula that includes variables, e.g., R, R₁-R₁₈, X, Y, Z, m, n, a, and b. Unless otherwise specified, each variable within such a formula is defined independently of other variables. Thus, if a group is said to be substituted, e.g., with 0-2 R*, then said group may be substituted with up to two R* groups and R* at each occurrence is selected independently from the definition of R*.

Combinations of substituents and/or variables are permissible only if such combinations result in stable compounds. A stable compound or stable structure is meant to imply a compound that is sufficiently robust to survive isolation from a reaction mixture, and subsequent formulation into an effective therapeutic agent.

The term “substituted” means that any one or more hydrogen atoms bound to the designated atom or group is replaced with a selection from the indicated group, provided that the designated atom's normal valence is not exceeded. Combinations of substituents and/or variables are permissible only if such combinations result in stable compounds or useful synthetic intermediates.

Substituents are named into the ring unless otherwise indicated. A dash (“—”) or a double bond (“═”) that is not between two letters or symbols indicates the point of attachment for a substituent. For example, —CONH₂ is attached through the carbon atom.

An “active agent” means a compound (including a compound disclosed herein), element, or mixture that when administered to a patient, alone or in combination with another compound, element, or mixture, confers, directly or indirectly, a physiological effect on the subject. The indirect physiological effect may occur via a metabolite or other indirect mechanism. The “active agent” may also potentiate or make more active another active agent. For example, the compounds of Formula I and Formula II potentiate the activity of other active agents when given in combination with another active agent, for example by lowering the effective dose of the other active agent.

An “aliphatic group” is a non-aromatic hydrocarbon group having the indicated number of carbon atoms. Aliphatic groups may be saturated, unsaturated, or cyclic.

“Alkyl” includes both branched and straight-chain saturated aliphatic hydrocarbon groups, having the specified number of carbon atoms. Thus, the term C₁-C₆alkyl includes alkyl groups having from 1 to about 6 carbon atoms. Examples of alkyl include, but are not limited to, methyl, ethyl, n-propyl, isopropyl, n-butyl, t-butyl, n-pentyl, and sec-pentyl. C₁-C₄alkyl includes alkyl groups having 1, 2, 3, or 4 carbon atoms.

“Alkoxy” is an alkyl group as defined above with the indicated number of carbon atoms attached through an oxygen bridge. Examples of alkoxy include, but are not limited to, methoxy, ethoxy, n-propoxy, i-propoxy, n-butoxy, 2-butoxy, t-butoxy, n-pentoxy, 2-pentoxy, 3-pentoxy, isopentoxy, neopentoxy, n-hexoxy, 2-hexoxy, 3-hexoxy, and 3-methylpentoxy.

“Haloalkyl” includes both branched and straight-chain saturated aliphatic hydrocarbon groups, having the specified number of carbon atoms, substituted with 1 or more halogen atoms, generally up to the maximum allowable number of halogen atoms. Thus, the term C₁-C₆haloalkyl includes haloalkyl groups having from 1 to about 6 carbon atoms. Examples of haloalkyl include, but are not limited to, trifluoromethyl, difluoromethyl, 2-fluoroethyl, chloromethyl, chloroethyl, and penta-fluoroethyl. C₁-C₂alkyl includes alkyl groups having 1 or 2 carbon atoms, substituted with 1 or more halogen atoms, generally up to the maximum allowable number of halogen atoms.

“Haloalkoxy” is an haloalkyl group as defined above with the indicated number of carbon atoms attached through an oxygen bridge. Examples of haloalkoxy include, but are not limited to, fluoromethoxy, trifluoromethoxy, fluoroethoxy, chloromethoxy, chloroethoxy, bromo-n-propoxy, bromo-i-propoxy, iodo-n-butoxy, iodo-2-butoxy, or chloro-n-pentoxy.

“Aryl” indicates aromatic groups containing only carbon in the aromatic ring or rings.

Typical aryl groups contain 1 to 3 separate, fused, or pendant rings and from 6 to about 18 ring atoms, without heteroatoms as ring members. When indicated, such aryl groups may be further substituted with carbon or non-carbon atoms or groups. Aryl groups include, for example, phenyl, naphthyl, including 1-naphthyl, 2-naphthyl, and bi-phenyl.

“Heteroaryl” is a stable monocyclic aromatic ring having the indicated number of ring atoms which contains from 1 to 3, or in some embodiments from 1 to 2, heteroatoms chosen from N, O, and S, with remaining ring atoms being carbon, or a stable bicyclic or tricyclic system containing at least one 5- to 7-membered aromatic ring which contains from 1 to 3, or in some embodiments from 1 to 2, heteroatoms chosen from N, O, and S, with remaining ring atoms being carbon. Monocyclic heteroaryl groups typically have from 5 to 7 ring atoms. In some embodiments bicyclic heteroaryl groups are 9- to 10-membered heteroaryl groups, that is, groups containing 9 or 10 ring atoms in which one 5- to 7-member aromatic ring is fused to a second aromatic or non-aromatic ring. When the total number of S and O atoms in the heteroaryl group exceeds 1, these heteroatoms are not adjacent to one another. It is preferred that the total number of S and O atoms in the heteroaryl group is not more than 2. It is particularly preferred that the total number of S and O atoms in the aromatic heterocycle is not more than 1. Examples of heteroaryl groups include, but are not limited to, oxazolyl, pyranyl, pyrazinyl, pyrazolopyrimidinyl, pyrazolyl, pyridizinyl, pyridyl, pyrimidinyl, pyrrolyl, quinolinyl, tetrazolyl, thiazolyl, thienylpyrazolyl, thiophenyl, triazolyl, benzo[d]oxazolyl, benzofuranyl, benzothiazolyl, benzothiophenyl, benzoxadiazolyl, dihydrobenzodioxynyl, furanyl, imidazolyl, indolyl, and isoxazolyl.

“Cycloalkyl” is a saturated hydrocarbon ring groups, having the specified number of carbon atoms, usually from 3 to 15 carbon atoms. Examples of cycloalkyl groups include cyclopropyl, cyclobutyl, cyclopentyl, or cyclohexyl as well as bridged, caged, or spiral saturated ring groups such as norbornane or adamantyl.

The term “heterocycloalkyl,” means a saturated ring group usually having 4- to 7-ring atoms with 1 or 2 ring atoms independently chosen from N, O, and S: Examples of heterocycloalkyl groups includes azepines, azetidinyl, morpholinyl, pyranyl, oxopiperidinyl, oxopyrrolidinyl, piperazinyl, piperidinyl, pyrrolidinyl, quinicludinyl, thiomorpholinyl, tetrahydropyranyl and tetrahydrofuranyl.

“Halo” or “halogen” as used herein refers to fluoro, chloro, bromo, or iodo.

“Pharmaceutical compositions” are compositions comprising at least one active agent, such as a compound of Formula I or Formula II, or a pharmaceutically acceptable salt, derivative, or solvate thereof, and at least one other excipient, such as a carrier. “Carriers” are any inactive materials, including excipients and diluents, which may be added to the pharmaceutical compositions including carriers and diluents. Pharmaceutical compositions meet the U.S. FDA's GMP (good manufacturing practice) standards for human or non-human drugs. Also included are any and all solvents, dispersion media, coatings, antibacterial and antifungal agents, and isotonic and absorption delaying agents. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions. A “pharmaceutically acceptable carrier” includes both one and more than one such carrier.

“Pharmaceutically acceptable salt” includes derivatives of the disclosed compounds wherein the parent compound is modified by making non-toxic acid or base salts thereof, and further refers to pharmaceutically acceptable hydrates or solvates of such compounds and such salts. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxylmaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like. Lists of additional suitable salts may be found, e.g., in G. Steffen Paulekuhn, et al., Journal of Medicinal Chemistry 2007, 50, 6665 and Handbook of Pharmaceutically Acceptable Salts: Properties, Selection and Use, P. Heinrich Stahl and Camille G. Wermuth Editors. Wiley-VCH, 2002.

“Prodrugs” means any compound which releases an active parent drug according to a Formula described herein in vivo when such prodrug is administered to a mammalian subject. Prodrugs may be prepared by modifying functional groups present in the compounds described herein in such a way that the modifications are cleaved, either in routine manipulation or in vivo, to the parent compounds. Prodrugs include compounds described herein wherein a hydroxy or amino group in a compound described herein is bonded to any group that may be cleaved in vivo to regenerate the free hydroxy or amino group, respectively. Examples of prodrugs include, but are not limited to esters (e.g., acetate, formate and benzoate derivatives), amides, guanidines, carbamates (e.g., N,N-dimethylaminocarbonyl) of hydroxy functional groups in compounds described herein. Preparation, selection and use of prodrugs is discussed in T. Higuchi and V. Stella, “Pro-drugs as Novel Delivery Systems,” Vol. 14 of the A.C.S. Symposium Series; “Design of Prodrugs,” ed. H. Bundgaard, Elsevier, 1985; and in Bioreversible Carriers in Drug Design, ed. Edward B. Roche, American Pharmaceutical Association and Pergamon Press, 1987, each of which are hereby incorporated by reference in their entirety.

A “metabolite” of a compound disclosed herein is a derivative of that compound that is formed when the compound is metabolized. The term “active metabolite” refers to a biologically active derivative of a compound that is formed when the compound is metabolized. The term “metabolized,” as used herein, refers to the sum of the processes (including, but not limited to, hydrolysis reactions and reactions catalyzed by enzymes, such as, oxidation reactions) by which a particular substance is changed by an organism. Information on metabolism may be obtained from The Pharmacological Basis of Therapeutics, 9th Edition, McGraw-Hill (1996). Metabolites of the compounds disclosed herein can be identified either by administration of compounds to a host and analysis of tissue samples from the host, or by incubation of compounds with hepatic cells in vitro and analysis of the resulting compounds. Both methods are well known in the art. In some embodiments, metabolites of a compound are formed by oxidative processes and correspond to the corresponding hydroxy-containing compound. In some embodiments, a compound is metabolized to pharmacologically active metabolites.

The term “carrier” applied to pharmaceutical compositions/combinations of the disclosure refers to a diluent, excipient, or vehicle with which an active compound is provided. A “pharmaceutically acceptable carrier” means a substance, e.g., excipient, diluent, or vehicle, that is useful in preparing a pharmaceutical composition that is generally safe, non-toxic and neither biologically nor otherwise undesirable, and includes a carrier that is acceptable for veterinary use as well as human pharmaceutical use. A “pharmaceutically acceptable carrier” includes both one and more than one such carrier.

A “patient” or a “subject” is a human or non-human animal in need of medical treatment. Medical treatment can include treatment of an existing condition, such as a disease or disorder or diagnostic treatment. In some embodiments the patient is a human patient.

“Providing” means giving, administering, selling, distributing, transferring (for profit or not), manufacturing, compounding, or dispensing.

“Treatment” or “treating” means providing an active compound to a patient in an amount sufficient to measurably reduce any existing condition or slow existing condition progression.

The term “therapeutically effective amount” of a compound of Formula I or II, or a pharmaceutical composition, means an amount effective, when administered to a patient, to provide a therapeutic benefit such as an amelioration of symptoms, decrease disease progression, or cause disease regression. Thus, a therapeutically effective amount of a compound or composition is also an amount sufficient to significantly reduce the indicia of the disease or condition being treated. A significant reduction is any detectable negative change that is statistically significant in a standard parametric test of statistical significance, such as Student's t-test, in which p<0.05.

“Administering” means giving, providing, applying, or dispensing by any suitable route. Administration of a combination of active agents includes administration of the combination in a single formulation or unit dosage form, administration of the individual active agents of the combination concurrently but separately, or administration of the individual active agents of the combination sequentially by any suitable route. The dosage of the individual active agents of the combination may require more frequent administration of one of the active agent(s) as compared to the other active agent(s) in the combination. Therefore, to permit appropriate dosing, packaged pharmaceutical products may contain one or more dosage forms that contain the combination of active agents, and one or more dosage forms that contain one of the combination of active agents, but not the other active agent(s) of the combination.

The term “room temperature” (rt or RT) used herein refers to a temperature of about 25° C.

### Chemical Description

The present disclosure provides novel compounds having soluble epoxide hydrolase inhibitory activity and can act as dual-target/multi-target agents. (3-Phenyl-oxiranyl)-acetic acid cyano-(6-methoxy-naphthalen-2-yl)-methyl ester (PHOME) has high aqueous stability and solubility, and is selected for the development of an fluorescence assay to determine human sEH (hsEH) inhibition as well as the IC₅₀ values of the potent inhibitors. The developed assay system is used to identify and validate new scaffolds as disclosed herein for the therapeutic inhibitory activity against soluble epoxide hydrolase.

In addition to compounds of Formula I shown above in the SUMMARY section, the disclosure includes the following particular embodiments of Formula I

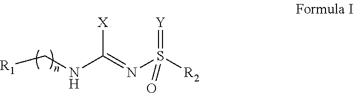

or a pharmaceutically acceptable ester, amide, solvate, salt, prodrug, or metabolite thereof, or a salt of such an ester, amide, prodrug, or metabolite, or a solvate of such an ester, amide, salt, prodrug, or metabolite, wherein:
n is 0 or 1;
R₁ is a cycloalkyl, and the cycloalkyl is unsubstituted or substituted with one to three substituents independently chosen from halogen, hydroxyl, amino, mono- or di-C₁-C₄alkylamino, C₁-C₆alkyl, C₁-C₃alkoxy, C₁-C₂haloalkyl, and C₁-C₂haloalkoxy;
R₂ is an aryl, and the aryl is unsubstituted or substituted with one to three substituents independently chosen from halogen, hydroxyl, cyano, nitro, amino, mono- or di-C₁-C₄alkylamino, C₁-C₆alkyl, C₁-C₃alkoxy, C₁-C₂haloalkyl, C₁-C₂haloalkoxy, —COOR₁₆, and —CONR₁₆R₁₆, wherein each R₁₆ is independently hydrogen or C₁-C₄ alkyl; or
R₂ is

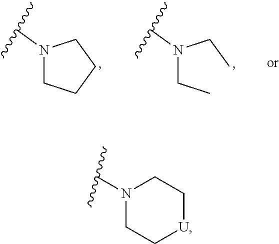

wherein U is NR₁₈, CH₂, O, S, or SO₂, wherein R₁₈ is hydrogen or C₁-C₆ alkyl; or
R₂ is —NR₁₁R₁₂, wherein R₁₁ is hydrogen, and R₁₂ is cycloalkyl, and the cycloalkyl is unsubstituted or substituted with one or more substituents independently chosen from hydrogen, halogen, hydroxyl, amino, mono- or di-C₁-C₄alkylamino, C₁-C₆alkyl, C₁-C₃alkoxy, C₁-C₂haloalkyl, and C₁-C₂haloalkoxy;
X is hydroxyl, or —N═CR₃R₄, wherein R₃ is —NHR, and R₄ is C₁-C₆alkyl, —SR, or —SeR, each R is independently hydrogen or C₁-C₆alkyl with one or more methylene groups optionally replaced by an oxygen; or R₃ and R₄ are joined to form a ring containing one additional heteroatom chosen from N, O, and S and the ring is unsubstituted or substituted with one or more substituents independently chosen from halogen, hydroxyl, cyano, nitro, amino, mono- or di-C₁-C₄alkylamino, C₁-C₃alkyl, C₁-C₃alkoxy, C₁-C₂haloalkyl, and C₁-C₂haloalkoxy.

Compounds of Formula I-A, which is a subformula of Formula I are also disclosed:

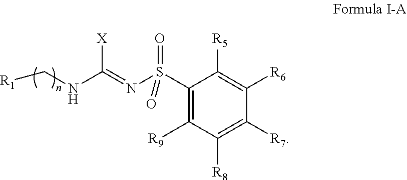

In Formula I-A, each of R₅ to R₉ is independently chosen from hydrogen, halogen, hydroxyl, cyano, nitro, amino, mono- or di-C₁-C₄alkylamino, C₁-C₆alkyl or C₁-C₄alkyl, C₁-C₃alkoxy, C₁-C₂haloalkyl, C₁-C₂haloalkoxy, —COOR₁₆, and —CONR₁₆R₁₆, wherein each R₁₆ is independently hydrogen or C₁-C₄ alkyl; and R₁, X, and n carry the same definitions as the corresponding variables in Formula I.

In addition to compounds of Formula II and Formula II-B shown above in the SUMMARY section, the disclosure includes the following particular embodiments of Formula II and Formula II-B

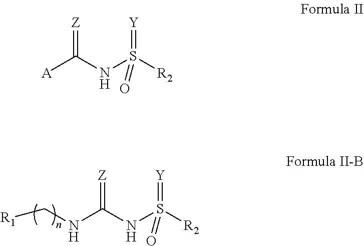

or a pharmaceutically acceptable ester, amide, solvate, salt, prodrug, or metabolite thereof, or a salt of such an ester, amide, prodrug, or metabolite, or a solvate of such an ester, amide, salt, prodrug, or metabolite, wherein:
A is

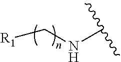

or —NR₂₁R₂₂;
n is 0 or 1;

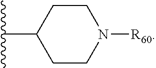

R₁ is a cycloalkyl or, and the cycloalkyl is unsubstituted or substituted with one to three substituents independently chosen from hydrogen, halogen, hydroxyl, amino, mono- or di-C₁-C₄alkylamino, C₁-C₆alkyl, C₁-C₃alkoxy, C₁-C₂haloalkyl, and C₁-C₂haloalkoxy;
R₆₀ is hydrogen, —C(O)—R₇₀, or —C(O)NHR₇₀, R₇₀ is C₁₋₆alkyl such as C₁-C₄alkyl, C₁-C₂alkyl, or methyl or cycloalkyl such as cyclohexyl, adamantyl, or memantyl;
R₂₁ and R₂₂ are each independently hydrogen, C₁₋₆alkyl, or cycloalkyl, or R₂₁, R₂₂, together with the nitrogen atom in —NR₂₁R₂₂ form a five membered or six membered heterocycloalkyl, which is unsubstituted or substituted with one or more substituents independently chosen from halogen, hydroxyl, amino, (mono- or di-C₁-C₆alkylamino)C₀-C₄alkyl, C₁-C₆alkyl, C₁-C₆alkoxy, C₁-C₆haloalkyl, and C₁-C₆haloalkoxy;
R₂ is an aryl, and the aryl is unsubstituted or substituted with one to three substituents independently chosen from halogen, hydroxyl, cyano, nitro, amino, mono- or di-C₁-C₄alkylamino, C₁-C₆alkyl, C₁-C₃alkoxy, C₁-C₂haloalkyl, and C₁-C₂haloalkoxy, —COOR₁₆, and —CONR₁₆R₁₆, wherein each R₁₆ is independently hydrogen or C₁-C₄ alkyl; or
R₂ is —NR₁₁R₁₂, wherein R₁₁ is hydrogen, and R₁₂ is cycloalkyl, and the cycloalkyl is unsubstituted or substituted with one to three substituents independently chosen from hydrogen, halogen, hydroxyl, amino, mono- or di-C₁-C₄alkylamino, C₁-C₆alkyl, C₁-C₃alkoxy, C₁-C₂haloalkyl, and C₁-C₂haloalkoxy; or
R₂ is

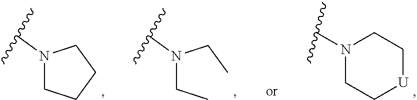

wherein U is NR₁₈, CH₂, O, S, or SO₂, wherein R₁₈ is hydrogen or C₁-C₆ alkyl; or
Y is O or NH; and
Z is S or O.

Compounds of Formula II-A and Formula II-C, which are subformulas of Formula II are also disclosed:

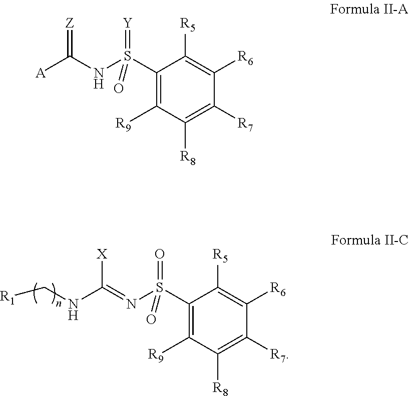

In Formula II-A and II-C, each of R₅ to R₉ is independently chosen from hydrogen, halogen, hydroxyl, cyano, nitro, amino, C₁-C₆alkyl or C₁-C₄alkyl, C₁-C₃alkoxy, C₁-C₂haloalkyl, C₁-C₂haloalkoxy, —COOR₁₆, or —CONR₁₆R₁₆, wherein each R₁₆ is independently hydrogen or C₁-C₄ alkyl; and R₁, X, and n carry the same definitions as the corresponding variables in Formula II.

Formulae I, I-A, II, II-A, II-B, and II-C include embodiments in which the variables, e.g.

R₁, R₂, R₃, R₄, R₅, R₆, R₇, R₈, R₉, n, X, and Y carry the definitions set forth below. The variable definitions can be combined in any combination that results in a stable compound.
(a) R₁ is C₅-C₁₂cycloalky.
(b) R₁ is cyclohexyl, adamantyl, or memantyl.
(c) Y is O.
(d) R₅, R₆, R₈, and R₉ are hydrogen.
(e) R₇ is hydrogen, methyl, ethyl, propyl, t-butyl, methoxy, ethoxy, fluoro, chloro, trifluoromethyl, trifluoromethoxy, cyano, or nitro.
(f) one of R₅ and R₆ is methyl, ethyl, propyl, t-butyl, methoxy, ethoxy, fluoro, chloro, trifluoromethyl, trifluoromethoxy, cyano, or nitro; and the other of R₅ and R₆ is hydrogen, and R₇, R₈, and R₉ are hydrogen.
(f) X is hydroxyl,

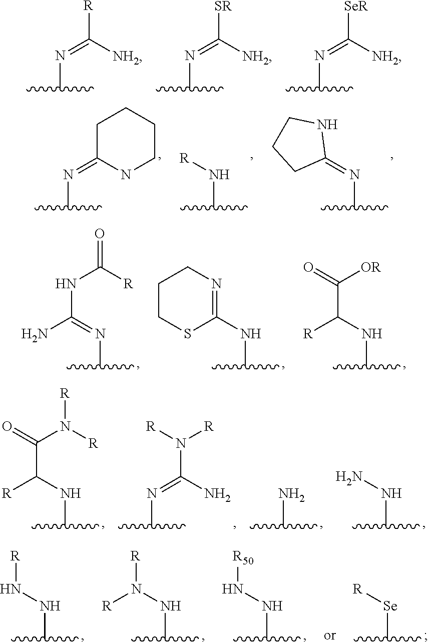

wherein R is hydrogen or C₁-C₆alkyl such as C₁-C₄alkyl, C₁-C₂alkyl, or methyl, and R₅₀ is phenyl.
(g) n is 0 to 1.
(h) R₂ is a substituted or unsubstituted phenyl, or a substituted or unsubstituted naphthyl.
(i) R₂ is —NR₁₁R₁₂, wherein R₁₁ is hydrogen, and R₁₂ is a substituted or unsubstituted cycloalkyl such as cyclohexyl, adamantyl, or memantyl.
(j) R₂ is

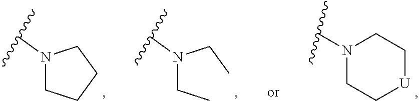

wherein U is NR₁₈, CH₂, O, S, or SO₂, wherein R₁₈ is hydrogen or C₁-C₆ alkyl.

The disclosure includes the following compounds of Formulae I and II shown below, and their pharmaceutically acceptable esters, amides, solvates, salts, prodrugs, or metabolites, or a salt of such an ester, amide, prodrug, or metabolite, or a solvate of such an ester, amide, salt, prodrug, or metabolite:

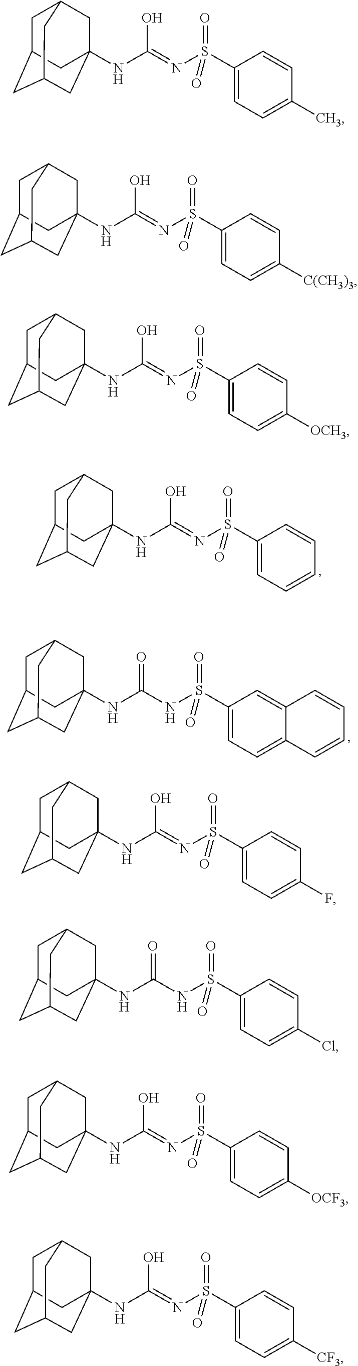

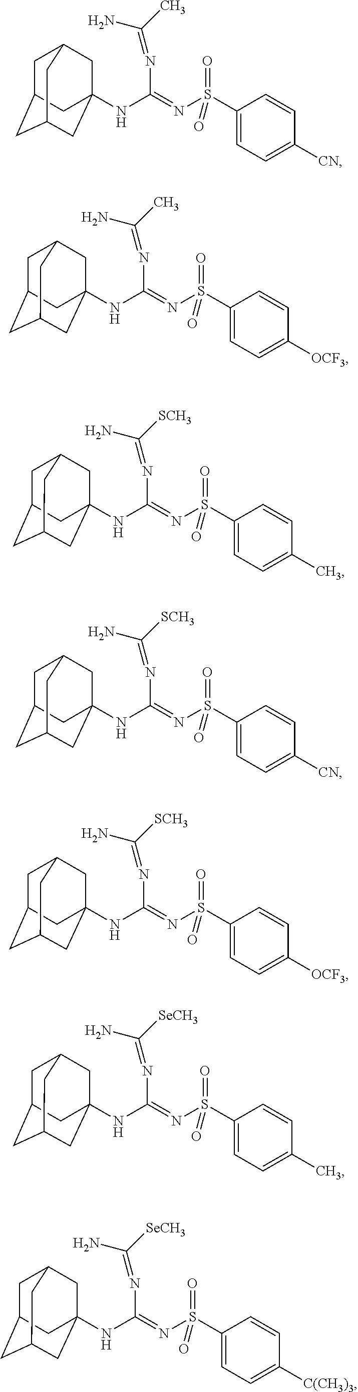

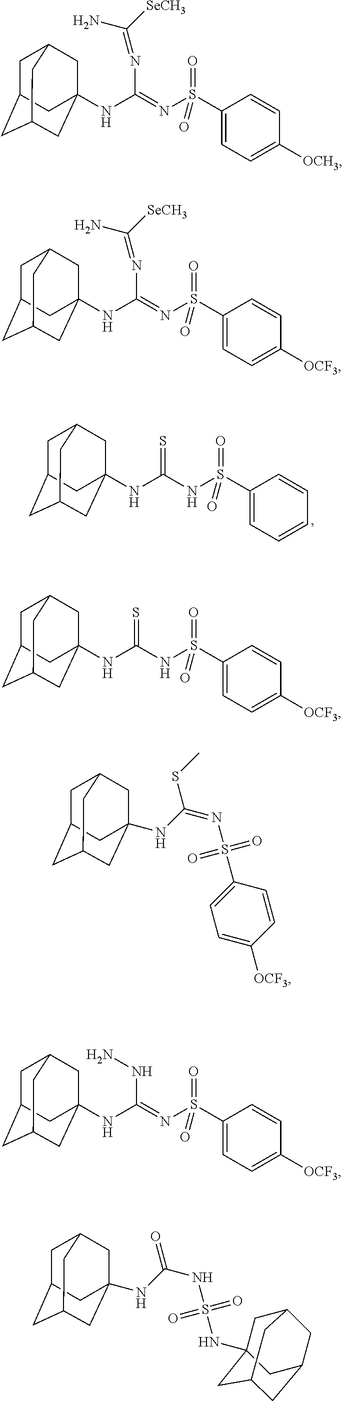

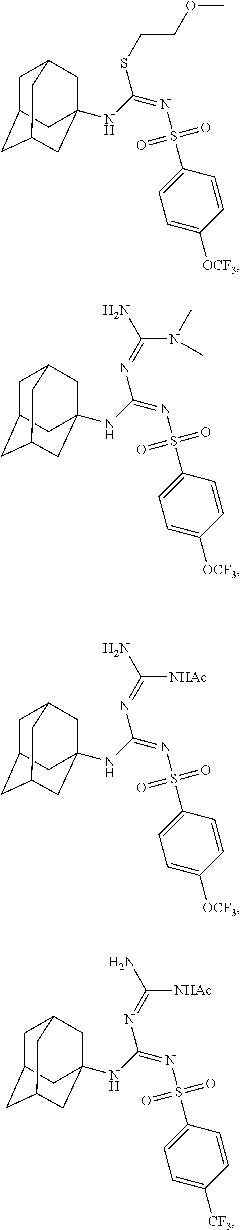 [SMILES: COCCS/C(=N/S(=O)(=O)c1ccc(OC(F)(F)F)cc1)NC12CC3CC(CC(C3)C1)C2 ; CN(C)/C(N)=N\C(=N\S(=O)(=O)c1ccc(OC(F)(F)F)cc1)NC12CC3CC(CC(C3)C1)C2 ; CC(=O)N/C(N)=N\C(=N\S(=O)(=O)c1ccc(OC(F)(F)F)cc1)NC12CC3CC(CC(C3)C1)C2 ; CC(=O)N/C(N)=N\C(=N\S(=O)(=O)c1ccc(C(F)(F)F)cc1)NC12CC3CC(CC(C3)C1)C2]

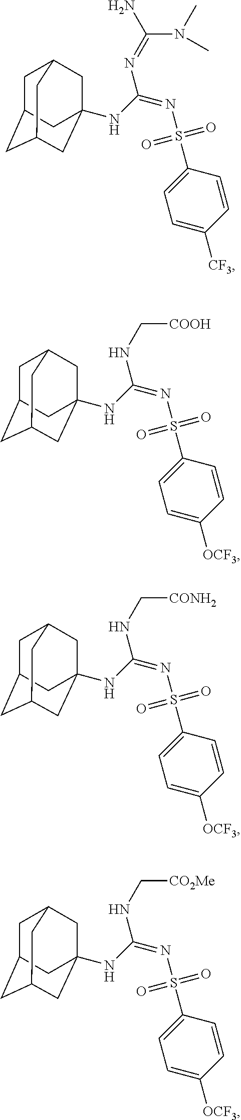 [SMILES: CN(C)/C(N)=N\C(=N\S(=O)(=O)c1ccc(C(F)(F)F)cc1)NC12CC3CC(CC(C3)C1)C2 ; O=C(O)CN/C(=N/S(=O)(=O)c1ccc(OC(F)(F)F)cc1)NC12CC3CC(CC(C3)C1)C2 ; NC(=O)CN/C(=N/S(=O)(=O)c1ccc(OC(F)(F)F)cc1)NC12CC3CC(CC(C3)C1)C2 ; COC(=O)CN/C(=N/S(=O)(=O)c1ccc(OC(F)(F)F)cc1)NC12CC3CC(CC(C3)C1)C2]

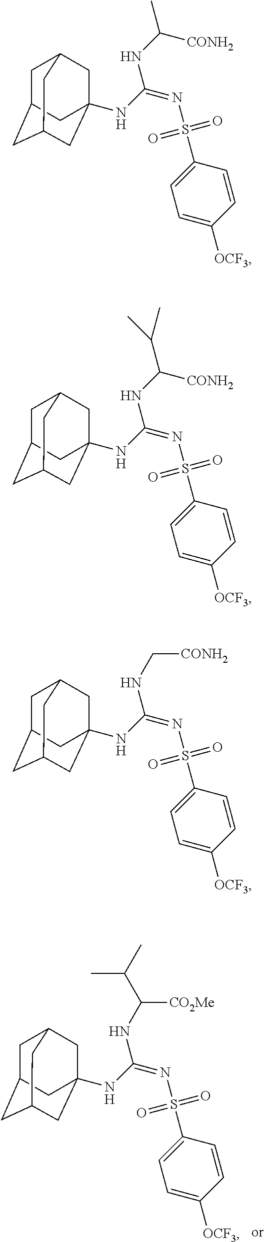 [SMILES: CC(N/C(=N/S(=O)(=O)c1ccc(OC(F)(F)F)cc1)NC12CC3CC(CC(C3)C1)C2)C(N)=O ; CC(C)C(N/C(=N/S(=O)(=O)c1ccc(OC(F)(F)F)cc1)NC12CC3CC(CC(C3)C1)C2)C(N)=O ; NC(=O)CN/C(=N/S(=O)(=O)c1ccc(OC(F)(F)F)cc1)NC12CC3CC(CC(C3)C1)C2 ; COC(=O)C(N/C(=N/S(=O)(=O)c1ccc(OC(F)(F)F)cc1)NC12CC3CC(CC(C3)C1)C2)C(C)C ; "or"]

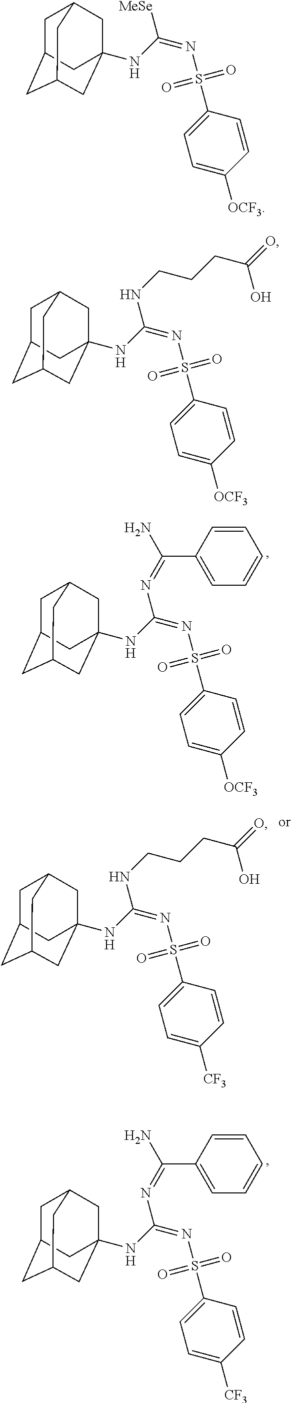

In the compounds, Me refers to —CH₃, and —NHAc refers to —NHC(═O)CH₃.

### Pharmaceutical Preparations

Compounds disclosed herein can be administered as the neat chemical, but are preferably administered as a pharmaceutical composition. Accordingly, the disclosure provides pharmaceutical compositions comprising a compound of Formula I or II, or a pharmaceutically acceptable ester, amide, solvate, salt, prodrug, metabolite thereof, or a salt of such an ester, amide, prodrug, or metabolite, or a solvate of such an ester, amide, salt, prodrug, metabolite (also referred to as “soluble epoxide hydrolase inhibitor”) together with at least one pharmaceutically acceptable carrier. The pharmaceutical composition/combination may contain a soluble epoxide hydrolase inhibitor as the only active agent or may be combined with one or more additional active agents. In certain embodiments the pharmaceutical composition is in a dosage form that contains from about 0.1 milligrams (mg) to about 4000 mg, from 0.1 mg to 3000 mg, from about 10 mg to about 2000 mg, from about 25 mg to about 1200 mg, or from about 50 mg to about 1000 mg of a soluble epoxide hydrolase inhibitor described herein.

Compounds disclosed herein may be administered orally, topically, parenterally, by inhalation or spray, sublingually, transdermally, via buccal administration, or by other means routine in the art for administering pharmaceutical compositions. The pharmaceutical composition may be formulated as any pharmaceutically useful form, e.g., as an aerosol, a cream, a gel, a pill, a capsule, a tablet, a syrup, a transdermal patch, or an ophthalmic solution.

Some dosage forms, such as tablets and capsules, are subdivided into suitably sized unit doses containing appropriate quantities of the active components, e.g., an effective amount to achieve the desired purpose.

Carriers include excipients and diluents and must be of sufficiently high purity and sufficiently low toxicity to render them suitable for administration to the patient being treated. The carrier can be inert or it can possess pharmaceutical benefits of its own. The amount of carrier employed in conjunction with the compound is sufficient to provide a practical quantity of material for administration per unit dose of the compound.

Classes of carriers include, but are not limited to binders, buffering agents, coloring agents, diluents, disintegrants, emulsifiers, flavorants, glidants, lubricants, preservatives, stabilizers, surfactants, tableting agents, and wetting agents. Some carriers may be listed in more than one class, for example vegetable oil may be used as a lubricant in some formulations and a diluent in others. Exemplary pharmaceutically acceptable carriers include sugars, starches, celluloses, powdered tragacanth, malt, gelatin; talc, and vegetable oils. Optional active agents may be included in a pharmaceutical composition, which do not substantially interfere with the activity of the compound of the present invention.

The pharmaceutical compositions/combinations can be formulated for oral administration. These compositions contain between 0.1 and 99 weight % (wt. %) of a soluble epoxide hydrolase inhibitor as described herein and usually at least about 5 wt. % of a soluble epoxide hydrolase inhibitor. Some embodiments contain from about 25 wt. % to about 50 wt. % or from about 5 wt. % to about 75 wt. % of the soluble epoxide hydrolase inhibitor.

### Methods of Treatment

The disclosure provides methods of treating a hypertension, atherosclerosis, a pulmonary disease, diabetes, pain, inflammation, an immunological disorder, fibrosis, addictive disorders, or a condition treatable or preventable by inhibition of soluble epoxide hydrolase in a patient. The method comprises providing to a patient in need thereof a compound of Formula I or II or a pharmaceutically acceptable salt thereof or a pharmaceutical composition described herein.

A soluble epoxide hydrolase inhibitor as described herein may be the only active agent administered (monotherapy) or may be combined with one or more other active agents (combination, adjunct, or augmentation therapy).

Methods of treatment include providing certain dosage amounts of a soluble epoxide hydrolase inhibitor to a patient. Dosage levels of each compound of from about 0.1 mg to about 250 mg per kilogram of body weight per day are useful in the treatment of the above-indicated conditions (about 0.5 mg to about 15 g per patient per day). The amount of compound that may be combined with the carrier materials to produce a single dosage form will vary depending upon the patient treated and the particular mode of administration. Dosage unit forms will generally contain between from about 1 mg to about 1000 mg of each active compound. In certain embodiments 25 mg to 1000 mg, or 25 mg to 400 mg of a soluble epoxide hydrolase inhibitor is provided daily to a patient. Frequency of dosage may also vary depending on the compound used and the particular disease treated. However, for treatment of most diseases and disorders, a dosage regimen of 4 times daily or less can be used and in certain embodiments a dosage regimen of 1 or 2 times daily is used.

In an embodiment, the method further comprises administering to the patient in need thereof at least one additional therapeutic agent.

Such administration encompasses co-administration of these therapeutic agents in a substantially simultaneous manner, such as in a single dosage form having a fixed ratio of active ingredients or in separate dosage forms for each active ingredient. In addition, such administration also encompasses administration of each therapeutic agent in a sequential manner, either at approximately the same time or at different times. In either case, the treatment regimen will provide the beneficial effects of each therapeutic agent in the drug combination in treating the conditions or disorders described herein.

It will be understood, however, that the specific dose level for any particular patient will depend upon a variety of factors including the activity of the specific compound employed, the age, body weight, general health, sex, diet, time of administration, route of administration, and rate of excretion, drug combination and the severity of the particular disease undergoing therapy.

Methods of treatment provided herein are also useful for treatment of mammals other than humans, including for veterinary applications such as to treat horses and livestock e.g. cattle, sheep, cows, goats, swine and the like, and pets (companion animals) such as dogs and cats.

### EXAMPLES

### Abbreviations

ACN Acetonitrile
AUDA 12-(3-Adamantan-1-yl-ureido)dodecanoic acid
DCM Dichloromethane
DIPEA Diisopropylethylamine
ESI Electrospray ionization
HRMS High Resolution Mass Spectrometry
LC-MS Liquid Chromatography-Mass Spectrometry
Me Methyl
MS Mass Spectrometry
NMR Nuclear Magnetic Resonance
rt or RT Room temperature
PHOME (3-Phenyl-oxiranyl)-acetic acid cyano-(6-methoxy-naphthalen-2-yl)-methyl ester
TLC Thin layer chromatography
—CH₄H₄— In compounds 1e, 3e, 5e, and 6e, —C₄H₄— is fused to the benzene ring, forming a naphthalene with the structure

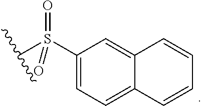 [SMILES: *S(=O)(=O)c1ccc2ccccc2c1]

### General Methods

All starting materials, building blocks, reagents, catalysts, dehydrating agents, and solvents utilized to synthesize the compounds of the present invention are either commercially available or can be produced by known organic synthesis methods in the art. Air sensitive reactions were carried out under dry nitrogen or argon atmosphere. The structures of final compounds, intermediates, and starting materials are confirmed by standard analytical methods, spectroscopic characterization e.g., MS, NMR. The purity of all the compounds was determined to be >95%, analyzed by LC-MS using isocratic elution of 1:1 acetonitrile and water both containing 0.1% formic acid with flow rate 1 mL/min with detection at 254 nm wavelength.

### Synthesis of Selected Compounds

General Procedure a for the Synthesis of Sulfonyl Carbamates (3a-n)

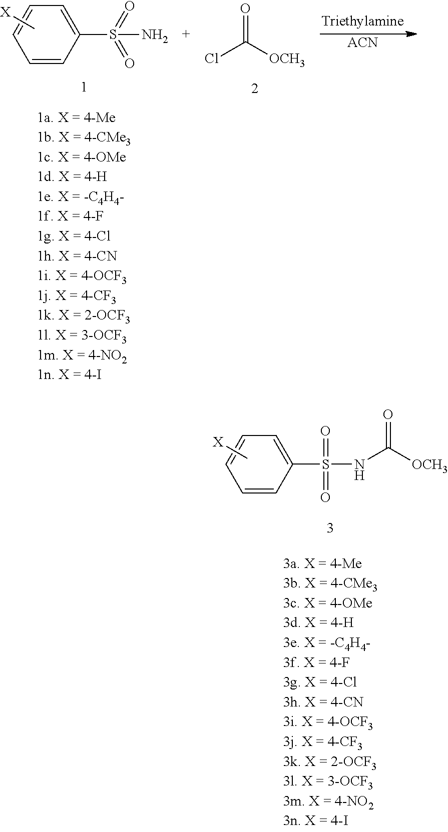

A sulfonamide of 1a-n (20 grams) was dissolved in dry ACN (120 mL). Triethylamine (2.5 equiv.) was added to the solution. Methyl chloroformate 2 (1.5 equiv.) was added dropwise to the reaction mixture in ice-cold condition. The reaction mixture was stirred at room temperature for 12 hours. Completion of the reaction was determined by thin layer chromatography. ACN was removed under vacuum. The residue was then dissolved in ethyl acetate, and the organic layer was extracted with saturated sodium bicarbonate solution. The aqueous portion was acidified with concentrated HCl to obtain a precipitate which was filtered, dried, and concentrated to provide a compound of 3a-n as a white solid.

### Example 1. Synthesis of methyl tosylcarbamate (3a)

Reaction of compound 1a (20 gm, 0.12 mol) with compound 2 (13.33 mL, 0.18 mol) was carried out according to general procedure A to afford compound 3a (48% yield). Analytical LCMS purity 99.42%, t_{R}=0.47 min; MS (ESI⁺) m/z 229.7 (M+H)⁺.

### Example 2. Synthesis of methyl ((4-(tert-butyl)phenyl)sulfonyl)carbamate (3b)

Reaction of compound 1b (20 gm, 0.07 mol) with compound 2 (10.89 mL, 0.11 mol) was carried out according to general procedure A to afford compound 3b (59% yield). Analytical LCMS purity 99.49%, t_{R}=0.95 min; MS (ESI⁺) m/z 271.7 (M+H)⁺.

### Example 3. Synthesis of methyl ((4-methoxyphenyl)sulfonyl)carbamate (3c)

Reaction of compound 1c (20 gm, 0.11 mol) with compound 2 (12.4 mL, 0.17 mol) was carried out according to general procedure A to afford compound 3c (54% yield). Analytical LCMS purity 99.62%, t_{R}=0.42 min; MS (ESI⁺) m/z 245.7 (M+H)⁺.

### Example 4. Synthesis of methyl (phenylsulfonyl)carbamate (3d)

Reaction of compound 1d (20 gm, 0.13 mol) with compound 2 (14.8 mL, 0.20 mol) was carried out according to general procedure A to afford compound 3d (47% yield). Analytical LCMS purity 99.61%, t_{R}=0.39 min; MS (ESI⁺) m/z 215.7 (M+H)⁺.

### Example 5. Synthesis of methyl (naphthalen-2-ylsulfonyl)carbamate (3e)

Reaction of compound 1e (20 gm, 0.08 mol) with compound 2 (11.49 mL, 0.15 mol) was carried out according to general procedure A to afford compound 3e (53% yield). Analytical LCMS purity 96.63%, t_{R}=2.67 min; MS (ESI⁺) m/z 385.5 (M+H)⁺.

### Example 6. Synthesis of methyl ((4-fluorophenyl)sulfonyl)carbamate (3f)

Reaction of compound if (20 gm, 0.11 mol) with compound 2 (13.25 mL, 0.17 mol) was carried out according to general procedure A to afford compound 3f (47% yield). Analytical LCMS purity 99.19%, t_{R}=0.44 min; MS (ESI⁺) m/z 233.7 (M+H)⁺.

### Example 7. Synthesis of methyl ((4-chlorophenyl)sulfonyl)carbamate (3g)

Reaction of compound 1g (20 gm, 0.10 mol) with compound 2 (12.13 mL, 0.15 mol) was carried out according to general procedure A to afford compound 3g (60% yield). Analytical LCMS purity 98.94%, t_{R}=0.53 min; MS (ESI⁺) m/z 249.7 (M+H)⁺.

### Example 8. Synthesis of methyl ((4-cyanophenyl)sulfonyl)carbamate (3h)

Reaction of compound 1h (20 gm, 0.11 mol) with compound 2 (12.76 mL, 0.16 mol) was carried out according to general procedure A to afford compound 3h (80% yield). Analytical LCMS purity 95.79%, t_{R}=0.53 min; MS (ESI⁺) m/z 240.7 (M+H)⁺.

### Example 9. Synthesis of methyl ((4-(trifluoromethoxy)phenyl)sulfonyl)carbamate (3i)

Reaction of compound 1i (20 gm, 0.08 mol) with compound 2 (9.64 mL, 0.12 mol) was carried out according to general procedure A to afford compound 3i (77% yield). Analytical LCMS purity 100.00%, t_{R}=0.54 min; MS (ESI⁺) m/z 299.7 (M+H)⁺.

### Example 10. Synthesis of methyl ((4-(trifluoromethyl)phenyl)sulfonyl)carbamate (3j)

Reaction of compound 1j (20 gm, 0.09 mol) with compound 2 (10.32 mL, 0.14 mol) was carried out according to general procedure A to afford compound 3j (70% yield).

### Example 11. Synthesis of methyl ((2-(trifluoromethoxy)phenyl)sulfonyl)carbamate (3k)

Reaction of compound 1k (20 gm, 0.08 mol) with compound 2 (9.64 mL, 0.12 mol) was carried out according to general procedure A to afford compound 3k (84% yield).

### Example 12. Synthesis of methyl ((3-(trifluoromethoxy)phenyl)sulfonyl)carbamate (31)

Reaction of compound 11 (20 gm, 0.08 mol) with compound 2 (9.64 mL, 0.12 mol) was carried out according to general procedure A to afford compound 31 (82% yield).

### Example 13. Synthesis of methyl ((4-nitrophenyl)sulfonyl)carbamate (3m)

Reaction of compound 1m (20 gm, 0.10 mol) with compound 2 (11.49 mL, 0.15 mol) was carried out according to general procedure A to afford compound 3m (96% yield). Analytical LCMS purity 99.63%, t_{R}=0.45 min; MS (ESI⁺) m/z 260.7 (M+H)⁺.

### General Procedure B for the Synthesis of 3-Hydroxy Adamantyl Ureas (4a, b, j):

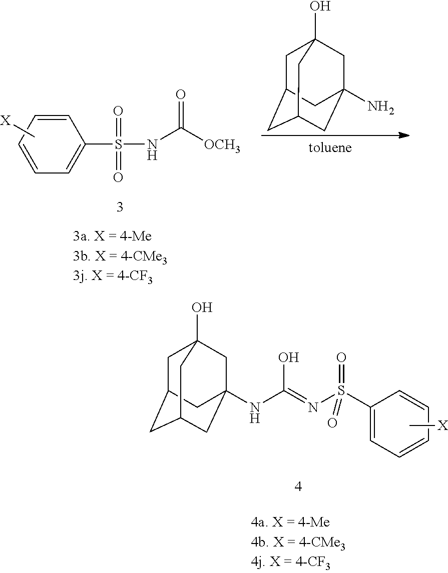

Sulfonyl carbamate 3 (5 gm) was dissolved in 50 mL toluene. To the solution, 3-aminoadamantan-1-ol (0.8 equiv.) was added, and the reaction mixture was refluxed for 4 hours.

Toluene was evaporated, and to it, isopropanol was added and triturated to afford a white solid product of one of compounds 4a-b, j.

### Example 14. Synthesis of N-(((1r,3s,5R,7S)-3-hydroxyadamantan-1-yl)carbamoyl)-4-methylbenzenesulfonamide (4a)

Compound 3a (5 gm, 21.81 mmol) was dissolved in 50 mL toluene. 3-aminoadamantan-1-ol was added to the solution, and the reaction was carried out according to general procedure B to obtain compound 4a (34% yield). Analytical LCMS purity 96.14%, t_{R}=0.46 min. HRMS m/z: [M+H]⁺ calculated for C₁₈H₂₅N₂O₄S, 365.1535; found, 365.1538.

### Example 15. Synthesis of 4-(tert-butyl)-N-(((1r,3s,5R,7S)-3-hydroxyadamantan-1-yl)carbamoyl)benzenesulfonamide (4b)

Compound 3b (5 gm, 18.43 mmol) was dissolved in 50 mL toluene. 3-aminoadamantan-1-ol was added to the solution, and the reaction was carried out according to general procedure B to afford compound 4b (31% yield). HRMS m/z: [M+H]⁺ calculated for C₂₁H₃₁N₂O₄S, 407.2005; found, 407.1999.

### Example 16. Synthesis of N-(((1r,3s,5R,7S)-3-hydroxyadamantan-1-yl)carbamoyl)-4-(trifluoromethyl)benzenesulfonamide (4j)

Compound 3j (5 gm, 17.65 mmol) was dissolved in 50 mL toluene. 3-aminoadamantan-1-ol was added to the solution, and the reaction was carried out according to general procedure B to afford compound 4j (41% yield).

General Procedure C for the Synthesis of Adamantyl Ureas (5a-1)

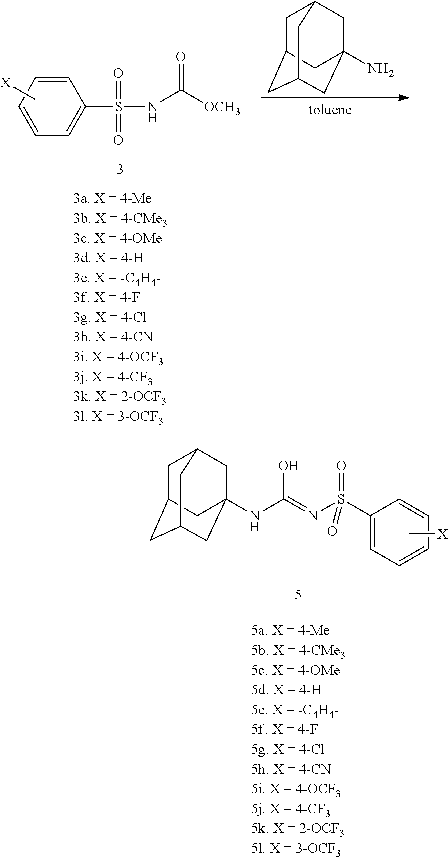

Sulfonyl carbamate 3 (5 gm) was dissolved in 50 mL toluene. To the solution, adamantyl amine (0.8 equiv.) was added, and the reaction mixture was refluxed for 4 hours. Toluene was evaporated, and to it, isopropanol was added and triturated to provide white a solid product of one of compounds 5a-1.

### Example 17. Synthesis of N-(((3s,5s,7s)-adamantan-1-yl)carbamoyl)-4-methylbenzenesulfonamide (5a)

Reaction of compound 3a (5 gm, 21.81 mmol) with 1-adamantylamine (2.54 gm, 16.78 mmol) was carried out following general procedure C to afford compound 5a (32% yield). Analytical LCMS purity 96.94%, t_{R}=1.87 min. HRMS m/z: [M+H]⁺ calculated for C₁₈H₂₅N₂O₃S, 349.1586; found, 349.1582.

### Example 18. Synthesis of N-(((3s,5s,7s)-adamantan-1-yl)carbamoyl)-4-(tert-butyl)benzenesulfonamide (5b)

Reaction of compound 3b (5 gm, 18.43 mmol) with 1-adamantylamine (2.23 gm, 14.74 mmol) was carried out following general procedure C to afford compound 5b (63% yield). Analytical LCMS purity 97.70%, t_{R}=2.98 min. HRMS m/z: [M+H]⁺ calculated for C₂₁H₃₁N₂O₃S, 391.2055; found, 391.2061.

### Example 19. Synthesis of N-(((3s,5s,7s)-adamantan-1-yl)carbamoyl)-4-methoxybenzenesulfonamide (5c)

Reaction of compound 3c (5 gm, 20.39 mmol) with 1-adamantylamine (2.37 gm, 15.68 mmol) was carried out following general procedure C to afford compound 5c (35% yield). Analytical LCMS purity 100%, t_{R}=1.57 min. HRMS m/z: [M+H]⁺ calculated for C₁₈H₂₅N₂O₄S, 365.1535; found, 365.1528.

### Example 20. Synthesis of N-(((3s,5s,7s)-adamantan-1-yl)carbamoyl)benzenesulfonamide (5d)

Reaction of compound 3d (5 gm, 23.23 mmol) with 1-adamantylamine (2.70 gm, 17.87 mmol) carried out following general procedure C to afford compound 5d (29% yield).

### Example 21. Synthesis of N-(((3s,5s,7s)-adamantan-1-yl)carbamoyl)-4-cyanobenzenesulfonamide (5h)

Reaction of compound 3h (5 gm, 20.81 mmol) with 1-adamantylamine (2.42 gm, 16.01 mmol) was carried out following general procedure C to afford compound 5h (33% yield). ¹H NMR (800 MHz; CDCl₃): δ 8.02 (d, J=6.6 Hz, 2H), 7.86 (d, J=6.3 Hz, 2H), 6.20 (s, 1H), 2.09 (s, 3H), 1.91 (s, 6H), 1.67 (d, J=13.7 Hz, 6H). HRMS m/z: [M+H]⁺ calculated for C₁₈H₂₂N₃O₃S, 360.1376; found, 360.1382.

### Example 22. Synthesis of N-(((3s,5s,7s)-adamantan-1-yl)carbamoyl)-4-(trifluoromethoxy)benzenesulfonamide (5i)

Reaction of compound 3i (5 gm, 16.71 mmol) with 1-adamantylamine (1.94 gm, 12.85 mmol) was carried out following general procedure C to afford compound 5i (37% yield). Analytical LCMS purity 94.87%, t_{R}=2.43 min. HRMS m/z: [M+H]⁺ calculated for C₁₈H₂₂F₃N₂O₄S, 419.1252; found, 419.1252.

### Example 23. Synthesis of N-(((3s,5s,7s)-adamantan-1-yl)carbamoyl)-4-(trifluoromethyl)benzenesulfonamide (5j)

Reaction of compound 3j (5 gm, 17.65 mmol) with 1-adamantylamine (2.05 gm, 13.56 mmol) was carried out following general procedure C to afford compound 5j (32% yield). Analytical LCMS purity 96.26%, t_{R}=2.29 min. HRMS m/z: [M+H]⁺ calculated for C₁₈H₂₂F₃N₂O₃S, 403.1303; found, 403.1307.

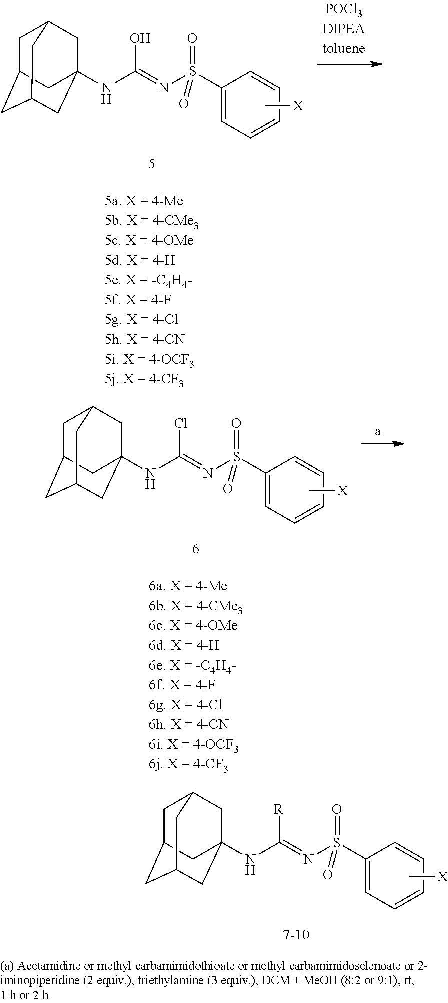

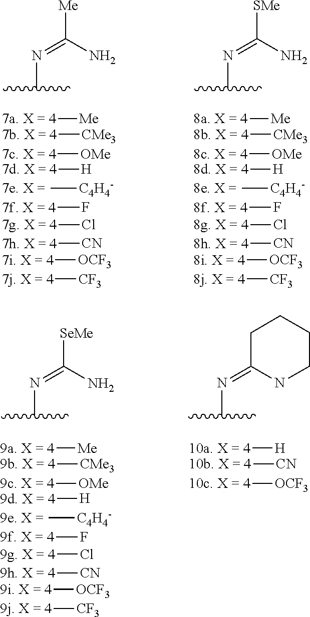

General Procedure D for the Synthesis of Acetimidamides (7a-j)

Compound 5 (0.5 gm, 1 equiv.) was dissolved in dry toluene (8 mL). To the solution was added N, N-diisopropylethylamine (2.5 equiv.) under N₂ atmosphere. Phosphorus oxychloride (POCl₃, 2 equiv.) was added to the reaction mixture under ice cold condition, and it was refluxed for 1 hour. Completion of the reaction was confirmed by thin layer chromatography. Intermediate 6 was not isolated. Toluene was evaporated, and a solution of acetamidine hydrochloride (2 equiv.) dissolved in an 10 mL mixture of DCM and methanol (8:2) was added dropwise to the reaction mixture in ice cold condition. Triethylamine (3 equiv.) was added to the mixture. The reaction mixture was stirred at room temperature for 2 hours. The solvent was removed under vacuum, and the residue was extracted with DCM and washed with a brine solution. The crude product was purified by silica gel column chromatography to provide a compound of one of 7a-j as a white solid.

### Example 24. Synthesis of N—N′-((3s,5s,7s)-adamantan-1-yl)-N′-tosylcarbamimidoyl) acetimidamide (7a)

Compound 5a (0.5 gm, 1.43 mmol) was reacted with POCl₃ (0.27 mL, 2.86 mmol) in dry toluene in the presence of N, N-diisopropylethylamine (0.62 mL, 3.58 mmol) as a base. Solvent was evaporated, and the intermediate formed was reacted with acetamidine hydrochloride (0.27 gm, 2.86 mmol) according to general procedure D to form compound 7a. Analytical LCMS purity 97.43%, t_{R}=2.18 min. HRMS m/z: [M+H]⁺ calculated for C₂₀H₂₉N₄O₂S, 389.2011; found, 389.2005.

### Example 25. Synthesis of N—N′-((3s,5s,7s)-adamantan-1-yl)-N′-((4-(tert-butyl)phenyl)sulfonyl)-carbamimidoyl)acetimidamide (7b)

Compound 5b (0.5 gm, 1.28 mmol) was reacted with POCl₃ (0.26 mL, 2.56 mmol) in dry toluene in the presence of N, N-diisopropylethylamine (0.56 mL, 3.2 mmol) as a base. Solvent was evaporated, and the intermediate formed was reacted with acetamidine hydrochloride (0.24 gm, 2.56 mmol) according to general procedure D to form compound 7b. Analytical LCMS purity 99.24%, t_{R}=2.78 min. HRMS m/z: [M+H]⁺ calculated for C₂₃H₃₅N₄O₂S, 431.2481; found, 431.2486. ¹H NMR (800 MHz; CDCl₃): δ 7.79 (d, J=8.4 Hz, 2H), 7.45 (d, J=8.4 Hz, 2H), 2.11 (s, 1H), 2.04 (s, 6H), 2.00 (s, 3H), 1.64 (t, J=9.7 Hz, 6H), 1.57 (s, 3H), 1.33 (d, J=4.0 Hz, 10H). Analytical LCMS purity 99.24%, t_{R}=2.78 min. HRMS m/z: [M+H]⁺ calculated for C₂₃H₃₅N₄O₂S, 431.2481; found, 431.2486.

### Example 26. Synthesis of N—N′-((3s,5s,7s)-adamantan-1-yl)-N′-((4-methoxyphenyl)sulfonyl)-carbamimidoyl)acetimidamide (7c)

Compound 5c (0.5 gm, 1.37 mmol) was reacted with POCl₃ (0.26 mL, 2.74 mmol) in dry toluene in the presence of N, N-diisopropylethylamine (0.60 mL, 3.4 mmol) as a base. Solvent was evaporated, and the intermediate formed was reacted with acetamidine hydrochloride (0.26 gm, 2.74 mmol) according to general procedure D to form compound 7c. Analytical LCMS purity 98.43%, t_{R}=1.30 min. HRMS m/z: [M+H]⁺ calculated for C₂₀H₂₉N₄O₃S, 405.1960; found, 405.1961.

### Example 27. Synthesis of N—N′-((3s,5s,7s)-adamantan-1-yl)-N′-((4-cyanophenyl)-sulfonyl)-carbamimidoyl)acetimidamide (7h)

Compound 5h (0.5 gm, 1.39 mmol) was reacted with POCl₃ (0.26 mL, 2.78 mmol) in dry toluene in the presence of N, N-diisopropylethylamine (0.61 mL, 3.48 mmol) as a base. Solvent was evaporated, and the intermediate formed was reacted with acetamidine hydrochloride (0.26 gm, 2.75 mmol) according to general procedure D to form compound 7h. ¹H NMR (800 MHz; CDCl₃): δ 7.97 (d, J=6.7 Hz, 2H), 7.75 (d, J=7.9 Hz, 2H), 2.14 (s, 1H), 2.07 (s, 6H), 2.01 (s, 3H), 1.99 (s, 3H), 1.65 (d, J=38.7 Hz, 6H). Analytical LCMS purity 99.22%, t_{R}=1.25 min. HRMS m/z: [M+H]⁺ calculated for C₂₀H₂₆N₅O₂S, 400.1807; found, 400.1804.

### Example 28. Synthesis of N—N′-((3s,5s,7s)-adamantan-1-yl)-N′-((4-(trifluoromethoxy)phenyl)-sulfonyl)carbamimidoyl)acetimidamide (7i)

Compound 5i (0.5 gm, 1.19 mmol) was reacted with POCl₃ (0.22 mL, 2.39 mmol) in dry toluene in the presence of N, N-diisopropylethylamine (0.52 mL, 2.97 mmol) as a base. Solvent was evaporated, and the intermediate formed was reacted with acetamidine hydrochloride (0.23 gm, 2.38 mmol) according to general procedure D to form compound 7i. ¹H NMR (800 MHz; CDCl₃): δ 7.94 (d, J=8.7 Hz, 2H), 7.28 (s, 2H), 2.11 (s, 3H), 2.06 (s, 3H), 1.92 (s, 6H), 1.71-1.64 (m, 6H). HRMS m/z: [M+H]⁺ calculated for C₂₀H₂₆F₃N₄O₃S, 459.1678; found, 459.1672.

### Example 29. Synthesis of N—N′-((3s,5s,7s)-adamantan-1-yl)-N′-((4-(trifluoromethyl)phenyl)sulfonyl)carbamimidoyl)acetimidamide (7j)

Compound 5j (0.5 gm, 1.24 mmol) was reacted with POCl₃ (0.23 mL, 2.48 mmol) in dry toluene in the presence of N, N-diisopropylethylamine (0.54 mL, 3.1 mmol) as a base. Solvent was evaporated, and the intermediate formed was reacted with acetamidine hydrochloride (0.23 gm, 2.48 mmol) according to general procedure D to form compound 7j. Analytical LCMS purity 96.98%, t_{R}=2.05 min. HRMS m/z: [M+H]⁺ calculated for C₂₀H₂₆N₄O₂F₃S, 443.1729; found, 443.1727.

General Procedure E for the Synthesis of Carbamimidothioates (8a-j)

Compound 5 (0.5 gm, 1 equiv.) was dissolved in dry toluene (8 mL). To the solution was added N, N-diisopropylethylamine (2.5 equiv.) under N₂ atmosphere. Phosphorus oxychloride (POCl₃, 2 equiv.) was added to the reaction mixture under ice cold condition, and it was refluxed for 1 hour. Completion of the reaction was confirmed by thin layer chromatography. Intermediate 6 was not isolated. Toluene was evaporated, and a solution of methyl carbamimidothioate (2 equiv.) dissolved in an 10 mL mixture of DCM and methanol (9:1) was added dropwise to the reaction mixture in ice cold condition. Triethylamine (3 equiv.) was added to the mixture. The reaction mixture was stirred at room temperature for 1 hour. The solvent was removed under vacuum, and the residue was extracted with DCM and washed with brine solution. The crude product was purified by silica gel column chromatography to afford one of compounds 8a-j as a solid.

### Example 30. Synthesis of methyl N—N′-((3s,5s,7s)-adamantan-1-yl)-N′-tosylcarbamimidoyl) carbamimidothioate (8a)

Compound 5a (0.5 gm, 1.43 mmol) was reacted with POCl₃ (0.27 mL, 2.86 mmol) in dry toluene in the presence of N, N-diisopropylethylamine (0.62 mL, 3.58 mmol) as a base. Solvent was evaporated, and the intermediate formed (6a) was reacted with methyl carbamimidothioate (0.62 gm, 2.86 mmol) according to general procedure E to form compound 8a. Analytical LCMS purity 99.43%, t_{R}=2.87 min. HRMS m/z: [M+H]⁺ calculated for C₂₀H₂₉N₄O₂S₂, 421.1732; found, 421.1739.

### Example 31. Synthesis of methyl N—N′-((3s,5s,7s)-adamantan-1-yl)-N′-((4-(tert-butyl)phenyl)sulfonyl)carbamimidoyl)carbamimidothioate (8b)

Compound 5b (0.5 gm, 1.28 mmol) was reacted with POCl₃ (0.26 mL, 2.56 mmol) in dry toluene in the presence of N, N-diisopropylethylamine (0.56 mL, 3.2 mmol) as a base. Solvent was evaporated, and the intermediate formed (6b) was reacted with methyl carbamimidothioate (0.56 gm, 2.56 mmol) according to general procedure E to form compound 8b. ¹H NMR (800 MHz; CDCl₃): δ 7.79 (d, J=8.2 Hz, 2H), 7.57 (s, 1H), 7.46 (d, J=8.2 Hz, 2H), 2.46 (s, 3H), 2.07 (s, 3H), 2.03 (s, 6H), 1.67-1.61 (m, 6H), 1.33 (s, 9H). Analytical LCMS purity 99.39%, t_{R}=3.98 min. HRMS m/z: [M+H]⁺ calculated for C₂₃H₃₅N₄O₂S₂, 463.2201; found, 463.2197.

### Example 32. Synthesis of methyl N—N′-((3s,5s,7s)-adamantan-1-yl)-N′-((4-methoxyphenyl)sulfonyl)carbamimidoyl)carbamimidothioate (8c)

Compound 5c (0.5 gm, 1.37 mmol) was reacted with POCl₃ (0.26 mL, 2.74 mmol) in dry toluene in the presence of N, N-diisopropylethylamine (0.60 mL, 3.4 mmol) as a base. Solvent was evaporated, and the intermediate formed (6c) was reacted with methyl carbamimidothioate (0.60 gm, 2.74 mmol) according to general procedure E to form compound 8c. Analytical LCMS purity 96.43%, t_{R}=2.63 min. HRMS m/z: [M+H]⁺ calculated for C₂₀H₂₉N₄O₃S₂, 437.1681; found, 437.1685.

### Example 33. Synthesis of methyl N—N′-((3s,5s,7s)-adamantan-1-yl)-N′-((4-cyanophenyl)sulfonyl)carbamimidoyl)carbamimidothioate (8h)

Compound 5h (0.5 gm, 1.39 mmol) was reacted with POCl₃ (0.26 mL, 2.78 mmol) in dry toluene in the presence of N, N-diisopropylethylamine (0.61 mL, 3.48 mmol) as a base. Solvent was evaporated, and the intermediate formed (6h) was reacted with methyl carbamimidothioate (0.62 gm, 2.84 mmol) according to general procedure E to form compound 8h. ¹H NMR (800 MHz; CDCl₃): δ 7.97 (d, J=6.8 Hz, 2H), 7.76 (d, J=7.7 Hz, 2H), 2.48 (s, 3H), 2.09 (s, 3H), 2.04 (s, 6H), 1.65 (d, J=54.9 Hz, 7H). HRMS m/z: [M+H]⁺ calculated for C₂₀H₂₆N₅O₂S₂, 432.1528; found, 432.1522.

### Example 34. Synthesis of N—N′-((3s,5s,7s)-adamantan-1-yl)-N′-((4-(trifluoromethyl)phenyl)sulfonyl)carbamimidoyl)carbamimidothioate (8j)

Compound 5j (0.5 gm, 1.24 mmol) was reacted with POCl₃ (0.23 mL, 2.48 mmol) in dry toluene in the presence of N, N-diisopropylethylamine (0.54 mL, 3.1 mmol) as a base. Solvent was evaporated, and the intermediate formed (6j) was reacted with methyl carbamimidothioate (0.54 gm, 2.48 mmol) according to general procedure E to form compound 8j. Analytical LCMS purity 99.79%, t_{R}=3.31 min. HRMS m/z: [M+H]⁺ calculated for C₂₀H₂₆N₄O₂F₃S₂, 475.1449; found, 475.1447.

General Procedure F for the Synthesis of Carbamimidoselenoates (9a-j)

Compound 5 (0.5 gm, 1 equiv.) was dissolved in dry toluene (8 mL). To the solution was added N, N-diisopropylethylamine (2.5 equiv.) under N₂ atmosphere. Phosphorus oxychloride (POCl₃, 2 equiv.) was added to the reaction mixture under ice cold condition, and it was refluxed for 1 hour. Completion of the reaction was confirmed by thin layer chromatography. Intermediate 6 was not isolated. Toluene was evaporated, and a solution of methyl carbamimidoselenoate (2 equiv.) dissolved in a 10 mL mixture of DCM and methanol (9:1) was added dropwise to the reaction mixture in ice cold condition. Triethylamine (3 equiv.) was added to the mixture. The reaction mixture was stirred at room temperature for 1 hour. The solvent was removed under vacuum, and the residue was extracted with DCM and washed with a brine solution. The crude product was purified by silica gel column chromatography to provide one of compounds 9a-j as a solid.

### Example 35. Synthesis of methyl N—N′-((3s,5s,7s)-adamantan-1-yl)-N′-tosylcarbamimidoyl) carbamimidoselenoate (9a)

Compound 5a (0.5 gm, 1.43 mmol) was reacted with POCl₃ (0.27 mL, 2.86 mmol) in dry toluene in the presence of N, N-diisopropylethylamine (0.62 mL, 3.58 mmol) as a base. Solvent was evaporated, and the intermediate formed (6a) was reacted with methyl carbamimidoselenoate (0.76 gm, 2.86 mmol) according to general procedure F to form compound 9a. Analytical LCMS purity 99.27%, t_{R}=0.71 min. HRMS m/z: [M+H]⁺ calculated for C₂₀H₂₉N₄O₂SSe, 469.1176; found, 469.1172.

### Example 36. Synthesis of methyl N—N′-((3s,5s,7s)-adamantan-1-yl)-N′-((4-(tert-butyl)phenyl)sulfonyl)carbamimidoyl)carbamimidoselenoate (9b)

Compound 5b (0.5 gm, 1.28 mmol) was reacted with POCl₃ (0.26 mL, 2.56 mmol) in dry toluene in the presence of N, N-diisopropylethylamine (0.56 mL, 3.2 mmol) as a base. Solvent was evaporated, and the intermediate formed (6b) was reacted with methyl carbamimidoselenoate (0.68 gm, 2.56 mmol) according to general procedure F to form compound 9b. Analytical LCMS purity 98.33%, t_{R}=3.75 min. HRMS m/z: [M+H]⁺ calculated for C₂₃H₃₅N₄O₂SSe, 511.1646; found, 511.1648.

### Example 37. Synthesis of methyl N—N′-((3s,5s,7s)-adamantan-1-yl)-N′-((4-cyanophenyl)-sulfonyl)carbamimidoyl)carbamimidoselenoate (9h)

Compound 5h (0.5 gm, 1.39 mmol) was reacted with POCl₃ (0.26 mL, 2.78 mmol) in dry toluene in the presence of N, N-diisopropylethylamine (0.61 mL, 3.48 mmol) as a base. Solvent was evaporated, and the intermediate formed was reacted with methyl carbamimidoselenoate (0.74 gm, 2.79 mmol) according to general procedure F to form compound 9h. ¹H NMR (800 MHz; CDCl₃): δ 7.98 (d, J=6.4 Hz, 2H), 7.76 (d, J=6.1 Hz, 2H), 2.37 (s, 3H), 2.09 (s, 3H), 2.03 (s, 6H), 1.98 (s, 1H), 1.66 (d, J=42.9 Hz, 6H). HRMS m/z: [M+H]⁺ calculated for C₂₀H₂₆N₅O₂SSe, 480.0972; found, 480.0966.

### Example 38. Synthesis of methyl N—N′-((3s,5s,7s)-adamantan-1-yl)-N′-((4-(trifluoromethyl)phenyl)sulfonyl)carbamimidoyl)carbamimidoselenoate (9j)

Compound 5j (0.5 gm, 1.24 mmol) was reacted with POCl₃ (0.23 mL, 2.48 mmol) in dry toluene in the presence of N, N-diisopropylethylamine (0.54 mL, 3.1 mmol) as a base. Solvent was evaporated, and the intermediate formed (6j) was reacted with methyl carbamimidoselenoate (0.66 gm, 2.48 mmol) according to general procedure F to form compound 9j. Analytical LCMS purity 94.80%, t_{R}=3.23 min. HRMS m/z: [M+H]⁺ calculated for C₂₀H₂₆N₄O₂F₃SSe, 523.0894; found, 523.0901.

### General Procedure G for the Synthesis of Compound 10a-c

Compound 5 (0.5 gm, 1 equiv.) was dissolved in dry toluene (8 mL). To the solution was added N, N-diisopropylethylamine (2.5 equiv.) under N₂ atmosphere. Phosphorus oxychloride (POCl₃, 2 equiv.) was added to the reaction mixture under ice cold condition, and it was refluxed for 1 hour. Completion of the reaction was confirmed by thin layer chromatography. Intermediate 6 was not isolated. Toluene was evaporated, and a solution of 2-iminopiperidine (2 equiv.) dissolved in a 10 mL mixture of DCM and methanol (9:1) was added dropwise to the reaction mixture in ice cold condition. Triethylamine (3 equiv.) was added to the mixture. The reaction mixture was stirred at room temperature for 2 hours. The solvent was removed under vacuum, and the residue was extracted with DCM and washed with a brine solution. The crude product was purified by silica gel column chromatography to afford one of compounds 10a-c as a solid.

### Example 39. Synthesis of N—((Z)-(((3s,5s,7s)-adamantan-1-yl)amino)(((E)-piperidin-2-ylidene)amino)methylene)-4-(trifluoromethoxy)benzenesulfonamide (10c)

Compound 5i (0.5 gm, 1.19 mmol) was reacted with POCl₃ (0.22 mL, 2.38 mmol) in dry toluene in the presence of N, N-diisopropylethylamine (0.52 mL, 2.98 mmol) as a base. Solvent was evaporated, and the intermediate formed (6i) was reacted with 2-iminopiperidine (0.32 gm, 2.38 mmol) according to general procedure G to form compound 10c. Analytical LCMS purity 97.19%, t_{R}=4.06 min.

General Procedure H for the Synthesis of Adamantyl Thioureas (11a-d)

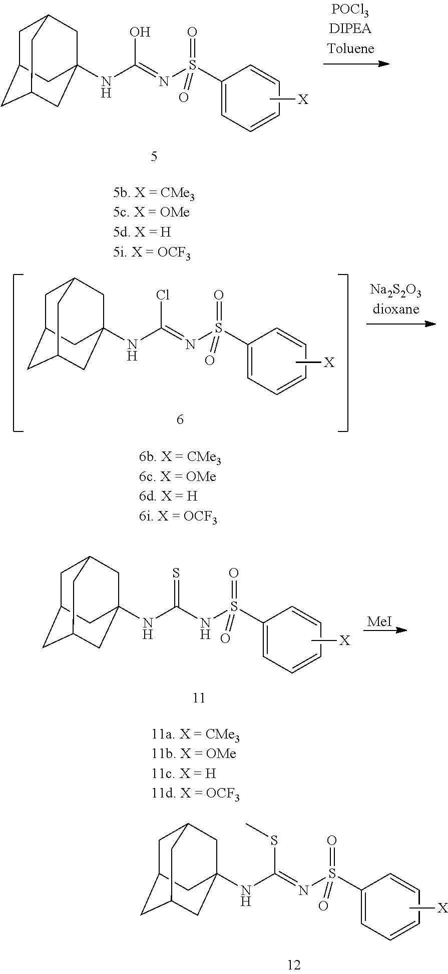

Compound 5 (0.5 gm, 1 equiv.) was dissolved in dry toluene (8 mL). To the solution was added N, N-diisopropylethylamine (2.5 equiv.) under N₂ atmosphere. Phosphorus oxychloride (POCl₃, 2 equiv.) was added to the reaction mixture under ice cold condition, and then the reaction mixture was refluxed for 1 hour. Completion of the reaction was confirmed by thin layer chromatography. Intermediate 6 was not isolated. Toluene was evaporated, and a solution of sodium thiosulfate (2 equiv.) dissolved in a 9 mL mixture of dioxane and water (8:1) was added dropwise to the residue. The reaction mixture was heated at 85-90° C. for 1-2 hours. The solvent was removed under vacuum, and the residue was extracted with DCM and washed with a brine solution. The crude product was purified by silica gel column chromatography to provide a compound of one of 11a-d as a white solid.

### Example 40. Synthesis of N-(((3s,5s,7s)-adamantan-1-yl)carbamothioyl)-4-(tert-butyl)benzenesulfonamide (11a)

Compound 5b (0.5 gm, 1.28 mmol) was reacted with POCl₃ (0.26 mL, 2.56 mmol) in dry toluene in the presence of N, N-diisopropylethylamine (0.56 mL, 3.2 mmol) as a base. Solvent was evaporated, and the intermediate formed (6b) was reacted with sodium thiosulfate (0.40 gm, 2.56 mmol) according to general procedure G to form compound 11a. ¹H-NMR (800 MHz; CDCl₃): δ 7.93 (s, 1H), 7.80 (d, J=8.0 Hz, 2H), 7.58 (d, J=8.0 Hz, 2H), 2.11 (s, 6H), 2.09 (s, 3H), 1.66 (s, 6H), 1.35 (s, 9H). ¹³C NMR (200 MHz; CDCl₃): δ 175.4, 158.6, 135.6, 127.2, 126.7, 55.9, 40.5, 31.2, 29.6. Analytical LCMS purity 98.26%, t_{R}=4.34 min. HRMS m/z: [M+H]⁺ calculated for C₂₁H₃₁N₂O₂S₂, 407.1827; found, 407.1833.

### Example 41. Synthesis of N-(((3s,5s,7s)-adamantan-1-yl)carbamothioyl)-4-methoxybenzenesulfonamide (11b)

Compound 5c (0.5 gm, 1.37 mmol) was reacted with POCl₃ (0.26 mL, 2.74 mmol) in dry toluene in the presence of N, N-diisopropylethylamine (0.60 mL, 3.4 mmol) as a base. Solvent was evaporated, and the intermediate formed (6c) was reacted with sodium thiosulfate (0.43 gm, 2.74 mmol) according to general procedure E to form compound 11b. ¹H-NMR (800 MHz; CDCl₃): δ 7.93 (s, 1H), 7.81 (d, J=8.0 Hz, 2H), 7.03 (d, J=8.0 Hz, 2H), 3.90 (s, 3H), 2.14 (s, 6H), 2.12 (s, 3H), 1.67 (s, 6H). ¹³C NMR (200 MHz; CDCl₃): δ 175.5, 164.3, 130.0, 129.6, 114.9, 56.0, 55.9, 40.6, 36.4, 29.6. Analytical LCMS purity 96.44%, t_{R}=2.93 min. HRMS m/z: [M+H]⁺ calculated for C₁₈H₂₅N₂O₃S₂, 381.1307; found, 381.1314.

### Example 42. Synthesis of N-(((3s,5s,7s)-adamantan-1-yl)carbamothioyl)benzenesulfonamide (11c)

Compound 5d (0.5 gm, 1.50 mmol) was reacted with POCl₃ (0.28 mL, 3.0 mmol) in dry toluene in the presence of N, N-diisopropylethylamine (0.65 mL, 3.75 mmol) as a base. Solvent was evaporated, and the intermediate formed (6d) was reacted with sodium thiosulfate (0.47 gm, 3.0 mmol) according to general procedure E to form compound 11c. ¹H-NMR (800 MHz; CDCl₃): δ 7.92 (s, 1H), 7.89 (d, J=8.0 Hz, 2H), 7.69 (s, 1H), 7.59 (d, J=8.0 Hz, 2H), 2.12 (s, 6H), 2.10 (s, 3H), 1.67 (s, 6H). ¹³C NMR (200 MHz; CDCl₃): δ 175.2, 138.6, 134.4, 129.7, 127.3, 55.9, 40.6, 36.3, 29.6. Analytical LCMS purity 100%, t_{R}=2.90 min. HRMS m/z: [M+H]⁺ calculated for C₁₇H₂₃N₂O₂S₂, 351.1201; found, 351.1199.

### Example 43. Synthesis of N-(((3s,5s,7s)-adamantan-1-yl)carbamothioyl)-4-(trifluoromethoxy)benzenesulfonamide (11d)

Compound 5i (0.5 gm, 1.19 mmol) was reacted with POCl₃ (0.22 mL, 2.38 mmol) in dry toluene in the presence of N, N-diisopropylethylamine (0.52 mL, 3.0 mmol) as a base. Solvent was evaporated, and the intermediate formed (6i) was reacted with sodium thiosulfate (0.38 gm, 2.38 mmol) according to general procedure E to form compound 11d. ¹H NMR (800 MHz; CDCl₃): δ 7.94 (d, J=8.3 Hz, 2H), 7.87 (s, 1H), 7.41 (d, J=8.4 Hz, 2H), 2.12 (s, 6H), 2.05 (s, 1H), 1.95 (s, 3H), 1.67 (d, J=15.9 Hz, 6H). ¹³C NMR (200 MHz; CDCl₃): δ 175.1, 153.4, 136.8, 129.6, 121.3, 42.8, 40.6, 36.3, 29.6. HRMS m/z: [M+H]⁺ calculated for C₁₈H₂₂F₃N₂O₃S₂, 435.1024; found, 435.1029.

### General Procedure I for the Synthesis of Compounds 13a-l

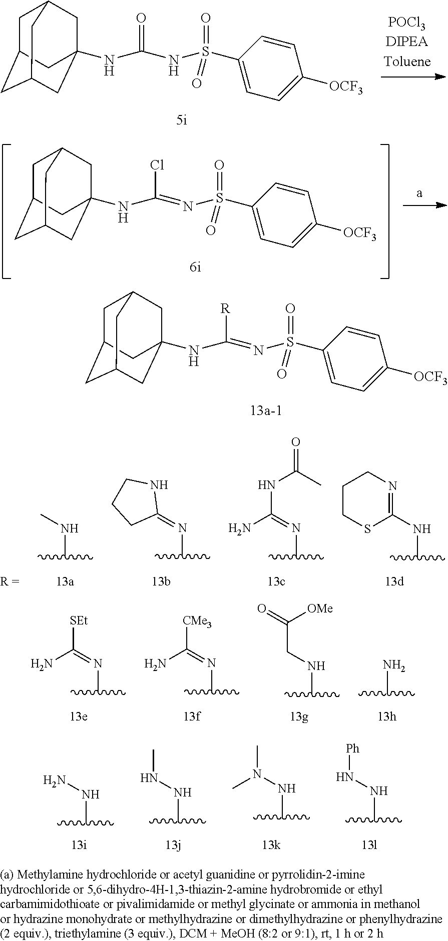

Compound 5i (0.5 gm, 1 equiv.) was dissolved in dry toluene (8 mL). To the solution was added N, N-diisopropylethylamine (2.5 equiv.) under N₂ atmosphere. Phosphorus oxychloride (POCl₃, 2 equiv.) was added to the reaction mixture under ice cold condition, and it was refluxed for 1 hour. Completion of the reaction was confirmed by thin layer chromatography. Intermediate 6i was not isolated. Toluene was evaporated, and a solution of appropriate amines (2 equiv.) dissolved in a 10 mL mixture of DCM and methanol (8:2 or 9:1) was added dropwise to the reaction mixture in ice cold condition. Triethylamine (3 equiv.) was added to the mixture. The reaction mixture was stirred at room temperature for 1 hour or 2 hours. The solvent was removed under vacuum, and the residue was extracted with DCM and washed with a brine solution. The crude product was purified by silica gel column chromatography to get one of compounds 13a-1 as a solid.

### Example 44. Synthesis of N—((E)-(((3s,5s,7s)-adamantan-1-yl)amino)(methylamino)methylene)-4-(trifluoromethoxy)benzenesulfonamide (13a)

Compound 5i (0.5 gm, 1.19 mmol) was reacted with POCl₃ (0.22 mL, 2.38 mmol) in dry toluene in the presence of N, N-diisopropylethylamine (0.52 mL, 2.98 mmol) as a base. Solvent was evaporated, and the intermediate formed (6i) was reacted with methylamine hydrochloride (0.16 gm, 2.38 mmol) according to general procedure I to form compound 13a. Analytical LCMS purity 97.72%, t_{R}=2.65 min.

### Example 45. Synthesis of N—((E)-N′—((Z)—N-((3s,5s,7s)-adamantan-1-yl)-N′-((4-(trifluoromethoxy)phenyl)sulfonyl)carbamimidoyl)carbamimidoyl)acetamide (13c)

Compound 5i (0.5 gm, 1.19 mmol) was reacted with POCl₃ (0.22 mL, 2.38 mmol) in dry toluene in the presence of N, N-diisopropylethylamine (0.52 mL, 2.98 mmol) as a base. Solvent was evaporated, and the intermediate formed (6i) was reacted with acetyl guanidine (0.24 gm, 2.38 mmol) according to general procedure I to afford compound 13c. Analytical LCMS purity 99.44%, t_{R}=3.04 min.

### Example 46. Synthesis of N—((Z)-(((3s,5s,7s)-adamantan-1-yl)amino)((5,6-dihydro-4H-1,3-thiazin-2-yl)amino)methylene)-4-(trifluoromethoxy)benzenesulfonamide (13d)

Compound 5i (0.5 gm, 1.19 mmol) was reacted with POCl₃ (0.22 mL, 2.38 mmol) in dry toluene in the presence of N, N-diisopropylethylamine (0.52 mL, 2.98 mmol) as a base. Solvent was evaporated, and the intermediate formed (6i) was reacted with 5,6-dihydro-4H-1,3-thiazin-2-amine hydrobromide (0.47 gm, 2.38 mmol) according to general procedure I to get compound 13d. Analytical LCMS purity 99.64%, t_{R}=4.08 min.

Additional compounds can be prepared via the following reaction schemes.

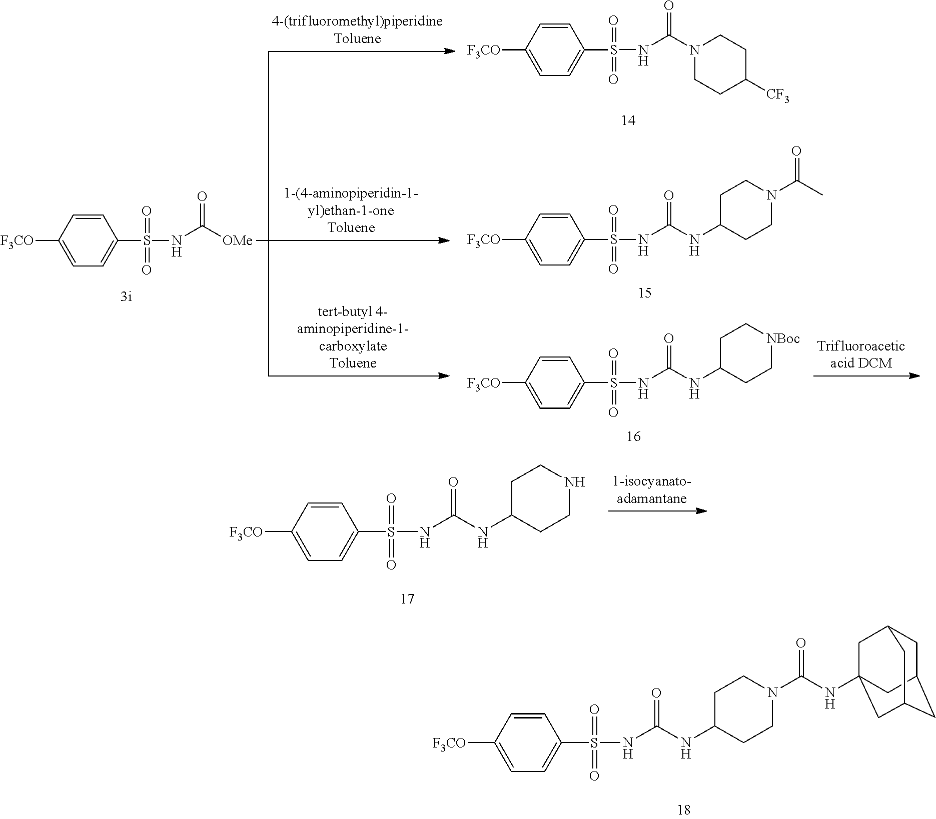

Additional characterization details on exemplary compounds are provided

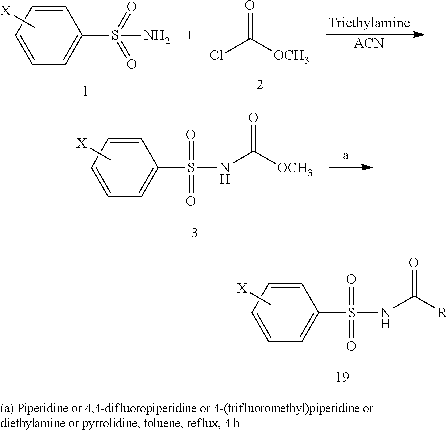

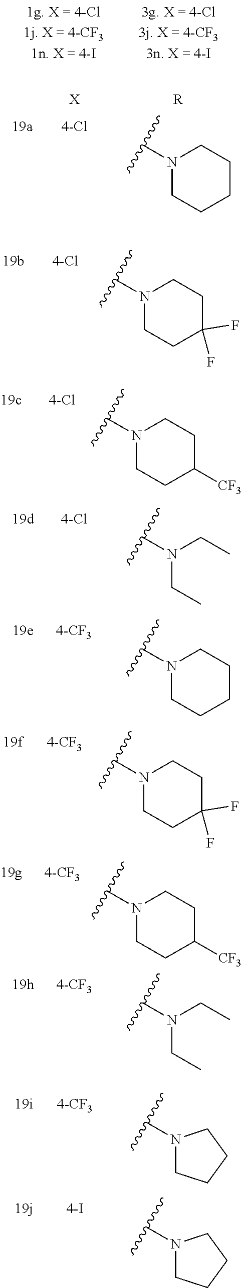

### Example 47. Synthesis of N-((4-chlorophenyl)sulfonyl)piperidine-1-carboxamide (19a)

To a solution of methyl ester (9 g) in toluene was added piperidine (3.96 mL), and the resulting slurry was refluxed for 3 hours. After cooling to the room temperature, the toluene was evaporated and the slurry was titrated with isopropyl alcohol (IPA) to obtain a white slurry. The white slurry was filtered and washed with a mixture of cold IPA and hexane (1:5) to give compound 19a (502 mg, 41%). ¹H NMR (500 MHz; CDCl₃) δ 8.01 (dd, J=3.3, 1.8 Hz, 2H), 7.51-7.49 (m, 2H), 3.33 (s, 4H), 1.56 (d, J=2.4 Hz, 7H). HRMS (C₁₂H₁₆N₂O₃SCl) [M+H]+: found m/z 303.0566, calculated 303.0570.

### Example 48. Synthesis of N-((4-(trifluoromethyl)phenyl)sulfonyl)pyrrolidine-1-carboxamide (19i)

To a solution of methyl ester (300 mg) in toluene was added amine (0.12 mL), and the resulting slurry was refluxed for 18 hours. After cooling to room temperature, toluene was evaporated, and the residue was dissolved into an aqueous 0.5 N NaOH solution. This mixture was extracted with diethyl ether. The aqueous layer was recovered and adjusted to pH of 1 with 0.5 N HCl. The precipitate was recovered by filtration to afford 200 mg (59%) of compound 19i. HRMS (C₁₂H₁₄N₂O₃F₃S) [M+H]+: found m/z 323.0673, calcd 323.0677.

### Example 49. Synthesis of N-((4-(trifluoromethyl)phenyl)sulfonyl)piperidine-1-carboxamide (19e)

To a solution of methyl ester (2.05 g) in toluene was added piperidine (680 mg), and the resulting slurry was refluxed for 3 hours. After cooling to room temperature, the toluene was evaporated and titrated with hexane and IPA to afford (503 mg, 42%) compound 19c. HRMS (C₁₃H₁₆N₂O₃F₃S) [M+H]+: found m/z 337.0837, calculated 337.0834.

### Example 50. Synthesis of 4-(Trifluoromethyl)-N-((4-(trifluoromethyl)phenyl)sulfonyl)piperidine-1-carboxamide (19g)

Sulfonamide methyl ester (448 mg, 1.58 mmol) was dissolved in a mixture of anhydrous toluene (2 mL) and amine (300 mg, 1.58 mmol) with triethanolamine (TEA, 0.22 mL, 1.58 mmol) and refluxed for 5 hours. The resulting solution was evaporated under reduced pressure, and the residue was dissolved into an aqueous NaOH solution (0.5 N). This mixture was extracted with diethyl ether. The aqueous layer was recovered and adjusted to pH of 1 with hydrochloric acid (0.5 N). The precipitate was recovered by filtration to afford 500 mg (78%) of compound 19g. ¹H NMR (500 MHz; CDCl₃) δ 8.20 (d, J=8.2 Hz, 2H), 7.82 (d, J=8.3 Hz, 2H), 4.07-4.05 (m, 1H), 2.86-2.81 (m, 2H), 2.22 (tdt, J=11.9, 8.0, 3.9 Hz, 1H), 1.92 (d, J=12.6 Hz, 2H), 1.71-1.58 (m, 2H), 1.56-1.48 (m, 2H). HRMS (C₁₄H₁₄N₂O₃F₆SNa) [M+Na]+: found m/z 427.0526, calculated 427.0527.

### Example 51. Synthesis of N-((4-cChlorophenyl)sulfonyl)-4-(trifluoromethyl)piperidine-1-carboxamide (19c)

Sulfonamide methyl ester (200 mg, 0.801 mmol) was dissolved in a mixture of anhydrous toluene (2 mL) and amine (152 mg, 0.801 mmol) with TEA (0.11 mL) and refluxed for 5 hours. The resulting solution was evaporated under reduced pressure, and the residue was dissolved into an aqueous NaOH solution (0.5 N). This mixture was extracted with diethyl ether. The aqueous layer was recovered and adjusted to pH of 1 with hydrochloric acid (0.5 N). The precipitate was recovered by filtration to afford 200 mg (67%) of compound 19c. ¹H NMR (500 MHz; CDCl₃) δ 8.00 (d, J=8.6 Hz, 2H), 7.51 (d, J=8.5 Hz, 2H), 4.08-4.03 (m, 2H), 2.85-2.79 (m, 2H), 2.25-2.18 (m, 1H), 1.91 (dd, J=13.7, 0.9 Hz, 2H), 1.54-1.49 (m, 3H). HRMS (C₁₃H₁₅N₂O₃F₃SCl) [M+H]+: found m/z 413.1069, calculated 413.1071.

### Example 52. Synthesis of N-((4-chlorophenyl)sulfonyl)-4,4-difluoropiperidine-1-carboxamide (19b)

Sulfonamide methyl ester (200 mg, 0.801 mmol) was dissolved in a mixture of anhydrous toluene (2 mL) and amine (126 mg, 0.801 mmol) with TEA (0.11 mL) and refluxed for 5 hours. The resulting solution was evaporated under reduced pressure, and the residue was dissolved into an aqueous NaOH solution (0.5 N). This mixture was extracted with diethyl ether. The aqueous layer was recovered and adjusted to pH of 1 with hydrochloric acid (0.5 N). The precipitate was recovered by filtration to afford 200 mg (74%) of compound 19b. HRMS (C₁₂H₁₄N₂O₃F₂SCl) [M+H]+: found m/z 339.0376, calculated 339.0382.

### Example 53. Synthesis of N-(diethylcarbamoyl)-4-(trifluoromethyl)benzenesulfonamide (19h)

To a solution of methyl ester (300 mg) in toluene was added amine (0.22 mL), and the resulting slurry was refluxed for 18 hours. After cooling to room temperature, toluene was evaporated, and the residue was dissolved into an aqueous 0.5 N NaOH solution. This mixture was extracted with diethyl ether. The aqueous layer was recovered and adjusted to pH of 1 with 0.5 N HCl. The precipitate was recovered by filtration to afford 174 mg (51%) of compound 19h. HRMS (C₁₂H₁₆N₂O₃F₃S) [M+H]+: found m/z 325.0828, calculated 325.0834.

### Example 54. Synthesis of 4-Chloro-N-(diethylcarbamoyl)benzenesulfonamide (19d)

To a solution of methyl ester (300 mg) in toluene was added amine (0.25 mL), and the resulting slurry was refluxed for 18 hours. After cooling to room temperature, the toluene was evaporated and titrated with hexane and IPA to afford the compound 19d (150 mg, 43%). HRMS (C₁₁H₁₆N₂O₃SCl) [M+H]+: found m/z 291.0568, calculated 291.0570.

### Biological Results

Fluorescence-based sEH inhibitor screening assay provided a convenient method for screening human epoxide hydrolase inhibitors. The assay utilized (3-phenyl-oxiranyl)-acetic acid cyano-(6-methoxy-naphthalen-2-yl)-methyl ester (PHOME) as substrate. When the epoxide moiety of PHOME was hydrolyzed by sEH, an intramolecular cyclization occurred which resulted in the release of a cyanohydrin under basic conditions. The cyanohydrin quickly decomposed into cyanide ion and the highly fluorescent 6-methoxy-2-naphthaldehyde which was analyzed using an excitation wavelength of 330 nm and emission wavelength of 465 nm after a 30-minute reaction time in darkness.

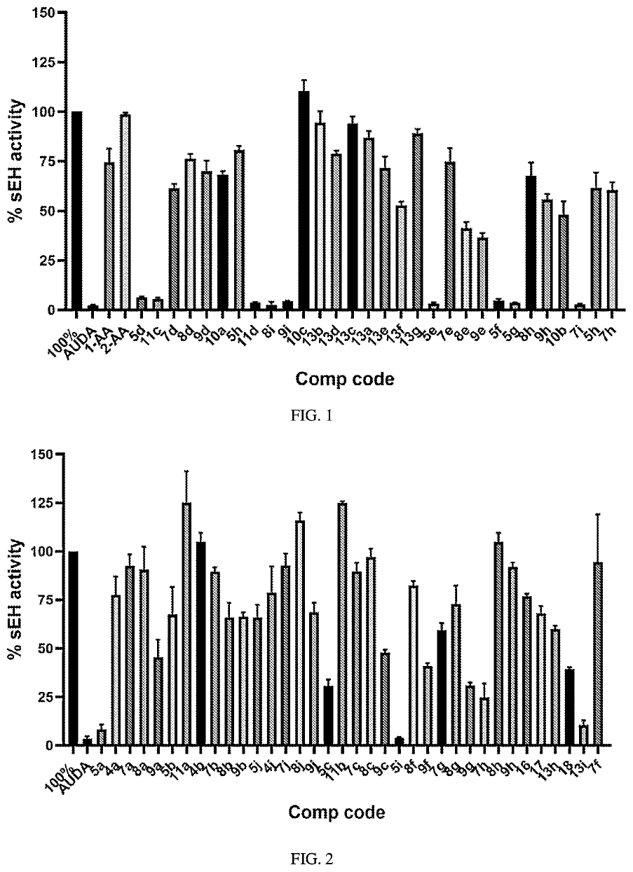

The above compounds were analyzed for sEH activity, and the results are shown in FIGS. 1-4. 1-AA is 1-adamantyl amine, 2-AA is 2-adamantyl amine.

Average fluorescence (AF) of the background wells, 100% initial activity wells with sEH but without any inhibitor added, a well with sEH and a known inhibitor AUDA added, and wells with sEH and each of the novel inhibitors added were determined. The background AF was subtracted from the 100% initial activity and inhibitor AFs. The following equation was used to calculate the % sEH activity, and the results are summarized in the Figure.

$$\% sEH activity = \{ \frac{Inhibited AF}{\% 100 initial activity} \} \times 100 \%$$

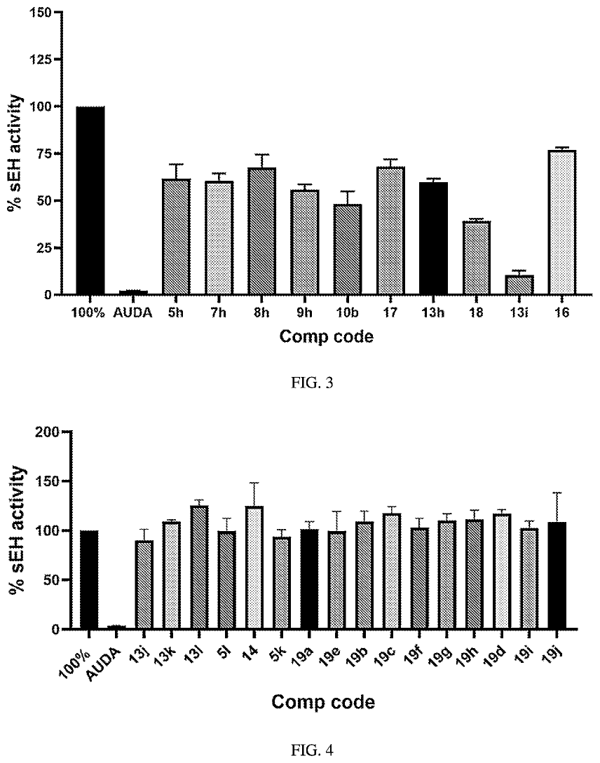

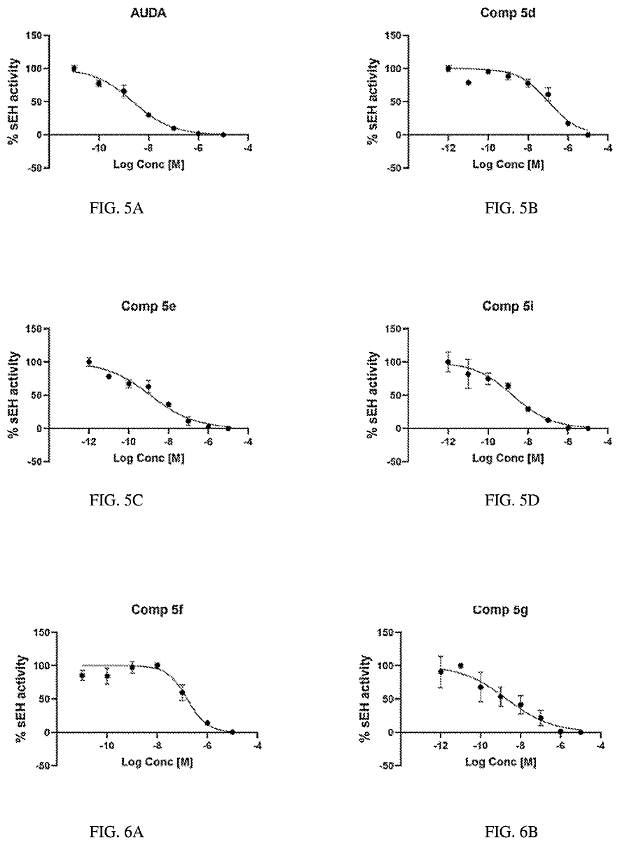

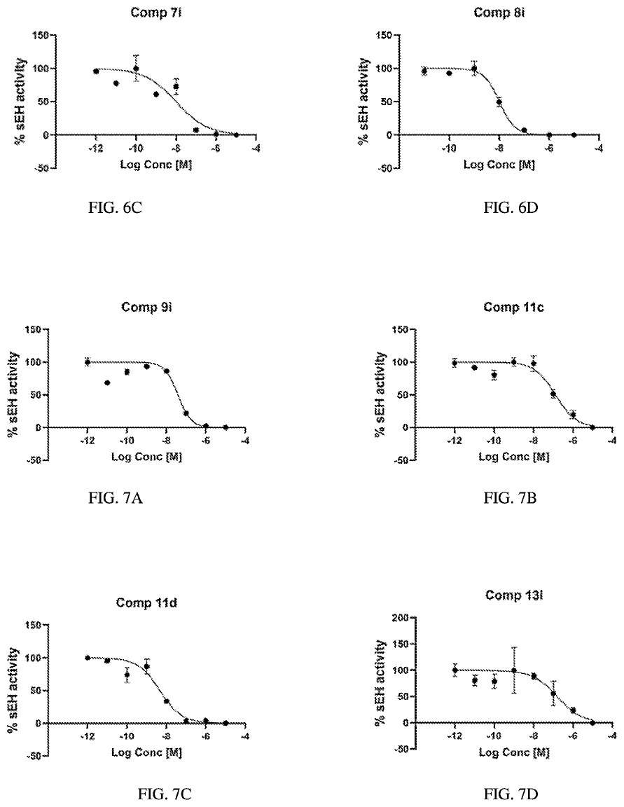

To calculate the half maximal inhibitory concentration (IC₅₀) values of the known inhibitor (AUDA) and selected potent inhibitors, dose-dependent fluorescence assay was performed. IC₅₀ values of these compounds are listed in the following Table 1. The graphs of sEH activity (%) versus log concentration (M) of AUDA, and selected inhibitors are shown in FIGS. 5A-5D, 6A-6D, and 7A-7D.

TABLE 1
sEH IC₅₀ values of the potent inhibitors

| Serial No. | Compound | sEH IC₅₀ (nM) |
|---|---|---|
| 1 | AUDA | 2.31 |
| 2 | 5d | 103.7 |
| 3 | 5e | 1.21 |
| 4 | 5f | 157.1 |
| 5 | 5g | 2.00 |
| 6 | 5i | 1.69 |
| 7 | 7i | 9.89 |
| 8 | 8i | 1.02 |
| 9 | 9i | 3.84 |
| 10 | 11c | 131.0 |
| 11 | 11d | 4.66 |
| 12 | 13i | 153.8 |

Notably, these compounds were found to exhibit significant inducible nitric oxide synthase (iNOS) inhibition in mouse cell line. Biochemical assay results are presented as the percent inhibition of specific binding or activity. The concentration of each compound was 10 μM. Significant responses (>50% inhibition or stimulation for Biochemical assays) were noted in the primary assays listed below in Table 2.

TABLE 2

| Serial No. | Compound | % iNOS inhibition |
|---|---|---|
| 1 | 8b | 52 |
| 2 | 9b | 95 |
| 3 | 9c | 94 |
| 4 | 9h | 93 |
| 5 | 9i | 87 |
| 6 | 9j | 90 |

In vitro ADME data is summarized in Table 3.

TABLE 3

| | Aqueous | | Caco-2 permeability | | | | | |
|---|---|---|---|---|---|---|---|---|
| | Solubility | | Aical to Basal | | Basal to Apical | | | |
| | @ pH 7.4 | | (Papp) in | | (Papp) in | | Efflux | |
| Comp. | (μg/mL) | | cm/sec (10⁻⁶) | | cm/sec (10⁻⁶) | | ratio | |
| 5e | 13 | | 19.9 | | 11.9 | | 0.6 | |
| 5i | 53 | | 14.9 | | 12.6 | | 0.8 | |
| | Plasma protein binding | | | | Metabolic stability | | | |
| | Human | | Mouse | | Human | | Mouse | |
| | % of | % of | % of | % of | Half life | % remaining | Half life | % remaining |
| Comp. | bound comp | stability | bound comp | stability | (min) | after 1 h | (min) | after 1 h |
| 5e | 100 | 99 | 98 | 88 | 33.4 | 45 | 2.2 | 0 |
| 5i | 100 | 99 | 98 | 106 | 106.3 | 80 | 4.9 | 0 |

The terms “a” and “an” do not denote a limitation of quantity, but rather denote the presence of at least one of the referenced item. The term “or” means “and/or.” The open-ended transitional phrase “comprising” encompasses the intermediate transitional phrase “consisting essentially of” and the close-ended phrase “consisting of.” Claims reciting one of these three transitional phrases, or with an alternate transitional phrase such as “containing” or “including” can be written with any other transitional phrase unless clearly precluded by the context or art. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the disclosure and does not pose a limitation on its scope unless otherwise claimed. No language in the specification should be construed as indicating any non-claimed element as essential to the practice of the invention as used herein. Unless defined otherwise, technical and scientific terms used herein have the same meaning as is commonly understood by one of skill in the art to which this disclosure belongs.

1. A compound of Formula I 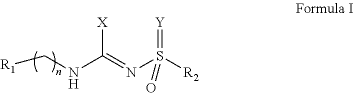
or a pharmaceutically acceptable ester, amide, solvate, salt, prodrug, or metabolite thereof, or a salt of such an ester, amide, prodrug, or metabolite, or a solvate of such an ester, amide, salt, prodrug, or metabolite, wherein:
n is 0 or 1;
R₁ is a cycloalkyl, and the cycloalkyl is unsubstituted or substituted with one or more substituents independently chosen from halogen, hydroxyl, amino, (mono- or di-C₁-C₆alkylamino)C₀-C₄alkyl, C₁-C₆alkyl, C₁-C₆alkoxy, C₁-C₆haloalkyl, and C₁-C₆haloalkoxy;
R₂ is an aryl or heteroaryl, and the aryl and the heteroaryl are unsubstituted or substituted with one or more substituents independently chosen from halogen, hydroxyl, cyano, nitro, amino, (mono- or di-C₁-C₆alkylamino)C₀-C₄alkyl, C₁-C₆alkyl, C₁-C₆alkoxy, C₁-C₆haloalkyl, C₁-C₆haloalkoxy, aryl, —COOR₁₆, —CONR₁₆R₁₆, —SO₂NR₁₆R₁₆, —B(OR₁₆)₃, —P(═O)(OR₁₆)₃, —NHNH₂, and triazolyl, wherein each R₁₆ is independently hydrogen or C₁-C₆ alkyl; or
R₂ is —NR₁₁R₁₂ or —N(CH₂)ₓ, wherein R₁₁ and R₁₂ are each independently hydrogen, C₁-C₆alkyl, or cycloalkyl, x is 4 to 6, and any one CH₂ in the —N(CH₂)ₓ is optionally replaced by NR₁₈, O, S, or SO₂, wherein R₁₈ is hydrogen or C₁-C₆ alkyl, and the cycloalkyl and —N(CH₂)ₓ are unsubstituted or substituted with one or more substituents independently chosen from halogen, hydroxyl, amino, (mono- or di-C₁-C₆alkylamino)C₀-C₄alkyl, C₁-C₆alkyl, C₁-C₆alkoxy, C₁-C₆haloalkyl, and C₁-C₆haloalkoxy;
X is —OR, —NHR, —SR(R₁₃)ₘ, —SeR(R₁₃)ₘ, —C(R)₃, —N(CH₂)_{b}SO₂R₁₄, —N(CH₂)_{b}COOR₁₄, —N(CH₂)_{b}COR₁₄, —N(CH₂)_{b}POR₁₄, —N(CH₂)_{b}BR₁₄, or —N═CR₃R₄, wherein each R is independently hydrogen, amino, cyano, or C₁-C₆alkyl with one or more methylene groups optionally replaced by an oxygen, R₁₃ is an oxo group, b is 0 to 6, m is 0, 1, or 2, R₁₄ is hydrogen or C₁-C₆alkyl, and R₃ and R₄ are each independently C₁-C₆alkyl, —SR(R₁₃)ₘ, —SeR(R₁₃)ₘ, —NHR, or R₃ and R₄ are joined to form a ring optionally containing one additional heteroatom chosen from N, O, and S and the ring is unsubstituted or substituted with one or more substituents independently chosen from halogen, hydroxyl, cyano, nitro, amino, (mono- or di-C₁-C₆alkylamino)C₀-C₄alkyl, C₁-C₆alkyl, C₁-C₆alkoxy, C₁-C₆haloalkyl, and C₁-C₆haloalkoxy; and
Y is O or NR₁₀, wherein R₁₀ is hydrogen or C₁-C₆alkyl.

2. The compound of claim 1, wherein X is hydroxyl.

3. The compound of claim 1, wherein:
X is —N═CR₃R₄, wherein R₃ is —NHR, and R₄ is C₁-C₆alkyl, —SR, or —SeR, each R is independently hydrogen or C₁-C₆alkyl; or R₃ and R₄ are joined to form a ring containing one additional heteroatom chosen from N, O, and S and the ring is unsubstituted or substituted with one or more substituents independently chosen from halogen, hydroxyl, cyano, nitro, amino, mono- or di-C₁-C₄alkylamino, C₁-C₃alkyl, C₁-C₃alkoxy, C₁-C₂haloalkyl, and C₁-C₂haloalkoxy.

4. The compound of claim 1, wherein X is 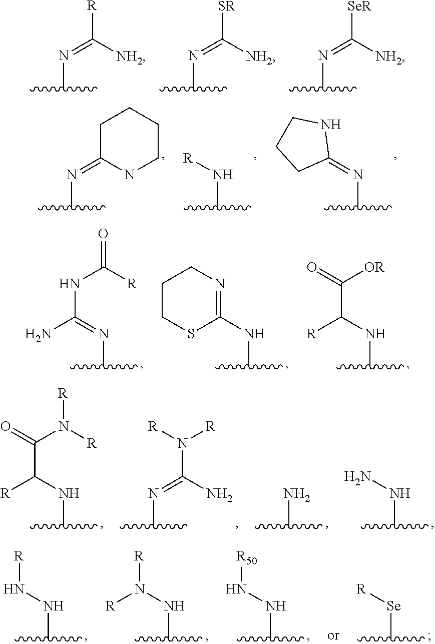
R is hydrogen or C₁-C₆alkyl;
R₅₀ is phenyl.

5. The compound of claim 1, wherein the compound of Formula I is a compound of Formula I-A: 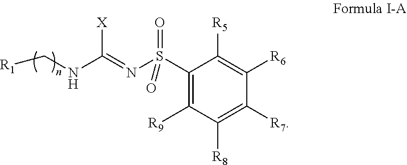
wherein each of R₅ to R₉ is independently chosen from hydrogen, halogen, hydroxyl, cyano, nitro, amino, mono- or di-C₁-C₄alkylamino, C₁-C₆alkyl, C₁-C₃alkoxy, C₁-C₂haloalkyl, C₁-C₂haloalkoxy, —COOR₁₆, and —CONR₁₆R₁₆, wherein each R₁₆ is independently hydrogen or C₁-C₄ alkyl.

6. The compound of claim 5, wherein R₅, R₆, R₈, and R₉ are hydrogen; and R₇ is hydrogen, methyl, ethyl, propyl, t-butyl, methoxy, ethoxy, fluoro, chloro, trifluoromethyl, trifluoromethoxy, cyano, or nitro.

7. The compound of claim 5, wherein one of R₅ or R₆ is methyl, ethyl, propyl, t-butyl, methoxy, ethoxy, fluoro, chloro, trifluoromethyl, trifluoromethoxy, cyano, or nitro, and the other of R₅ or R₆ is hydrogen; and R₇, R₈, and R₉ are hydrogen.

8. A compound of Formula II 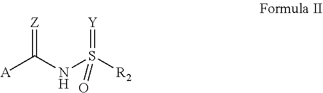
or a pharmaceutically acceptable ester, amide, solvate, salt, prodrug, or metabolite thereof, or a salt of such an ester, amide, prodrug, or metabolite, or a solvate of such an ester, amide, salt, prodrug, or metabolite, wherein:
A is 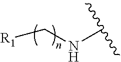
or —NR₂₁R₂₂;
n is 0 or 1;
R₁ is a cycloalkyl or 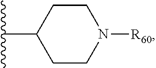
and the cycloalkyl is unsubstituted or substituted with one or more substituents independently chosen from halogen, hydroxyl, amino, (mono- or di-C₁-C₆alkylamino)C₀-C₄alkyl, C₁-C₆alkyl, C₁-C₆alkoxy, C₁-C₆haloalkyl, and C₁-C₆haloalkoxy;
R₆₀ is hydrogen, —C(O)—R₇₀, or —C(O)NHR₇₀, R₇₀ is C₁₋₆alkyl or cycloalkyl;
R₂₁ and R₂₂ are each independently hydrogen, C₁₋₆alkyl, or cycloalkyl, or R₂₁, R₂₂, together with the nitrogen atom in —NR₂₁R₂₂ form a five membered or six membered heterocycloalkyl, which is unsubstituted or substituted with one or more substituents independently chosen from halogen, hydroxyl, amino, (mono- or di-C₁-C₆alkylamino)C₀-C₄alkyl, C₁-C₆alkyl, C₁-C₆alkoxy, C₁-C₆haloalkyl, and C₁-C₆haloalkoxy;
R₂ is an aryl or heteroaryl, and the aryl and the heteroaryl are unsubstituted or substituted with one or more substituents independently chosen from halogen, hydroxyl, cyano, nitro, amino, (mono- or di-C₁-C₆alkylamino)C₀-C₄alkyl, C₁-C₆alkyl, C₁-C₆alkoxy, C₁-C₆haloalkyl, C₁-C₆haloalkoxy, aryl, —COOR₁₆, —CONR₁₆R₁₆, —SO₂NR₁₆R₁₆, —B(OR₁₆)₃, —P(═O)(OR₁₆)₃, —NHNH₂, and triazolyl, wherein each R₁₆ is independently hydrogen or C₁-C₆ alkyl; or
R₂ is —NR₁₁R₁₂ or —N(CH₂)ₓ, wherein R₁₁ and R₁₂ are each independently hydrogen, C₁-C₆alkyl, or cycloalkyl, x is 4 to 6, and any one CH₂ in the —N(CH₂)ₓ is optionally replaced by NR₁₈, O, S, or SO₂, wherein R₁₈ is hydrogen or C₁-C₆ alkyl, and the cycloalkyl and —N(CH₂)ₓ are unsubstituted or substituted with one or more substituents independently chosen from halogen, hydroxyl, amino, (mono- or di-C₁-C₆alkylamino)C₀-C₄alkyl, C₁-C₆alkyl, C₁-C₆alkoxy, C₁-C₆haloalkyl, and C₁-C₆haloalkoxy;
Y is O or NR₁₀, wherein R₁₀ is hydrogen or C₁-C₆alkyl; and
Z is O, S, or NR₁₅, wherein R₁₅ is hydrogen or C₁-C₆alkyl,
with the proviso that R₁ is not adamantyl when a is zero, Z and Y are O, and R₂ is 4-methylphenyl.

9. The compound of claim 8, wherein Z is S.

10. The compound of claim 8, wherein Z is O.

11. The compound of claim 8, wherein the compound of Formula II is a compound of Formula II-A: 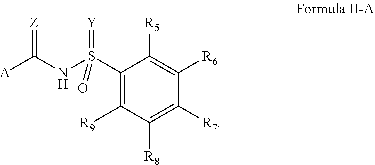
wherein each of R₅ to R₉ is independently chosen from hydrogen, halogen, hydroxyl, cyano, nitro, amino, mono- or di-C₁-C₄alkylamino, C₁-C₆alkyl, C₁-C₃alkoxy, C₁-C₂haloalkyl, C₁-C₂haloalkoxy, —COOR₁₆, and —CONR₁₆R₁₆, wherein each R₁₆ is independently hydrogen or C₁-C₄ alkyl.

12. The compound of claim 11, wherein R₅, R₆, R₈, and R₉ are hydrogen; and R₇ is hydrogen, methyl, ethyl, propyl, t-butyl, methoxy, ethoxy, fluoro, chloro, trifluoromethyl, trifluoromethoxy, cyano, or nitro.

13. The compound of claim 1 or claim 8, wherein R₂ is —NR₁₁R₁₂, R₁₁ is hydrogen, and R₁₂ is a substituted or unsubstituted cycloalkyl.

14. The compound of claim 13 wherein R₁₂ is cyclohexyl, adamantyl, or memantyl.

15. The compound of claim 1 or claim 8, wherein R₂ is a substituted or unsubstituted phenyl, or a substituted or unsubstituted naphthyl.

16. The compound of claim 1, wherein R₁ is C₅-C₁₂cycloalky.

17. The compound of claim 1, wherein R₁ is cyclohexyl, adamantyl, or memantyl.

18. The compound of claim 1, wherein Y is O.

19. The compound of claim 1, wherein n is 1.

20. The compound of claim 1, wherein n is 0.

21. A The compound of claim 1, wherein the compound is 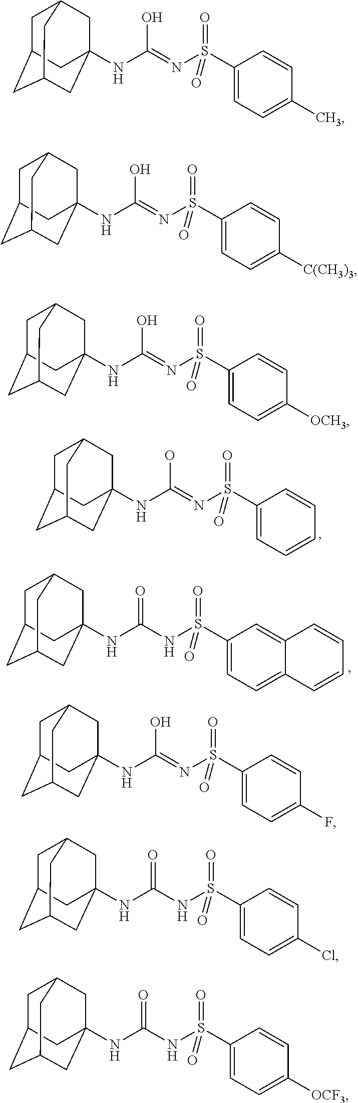 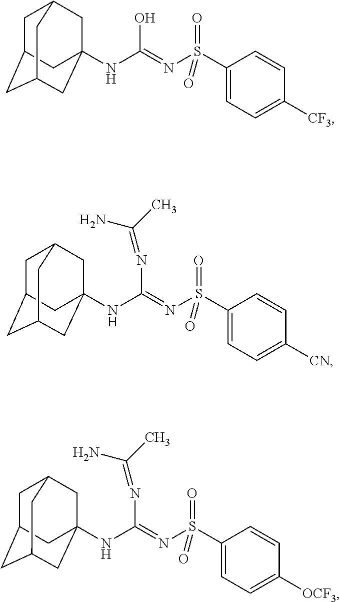 [SMILES: O=S(=O)(/N=C(\O)NC12CC3CC(CC(C3)C1)C2)c1ccc(C(F)(F)F)cc1 ; C/C(N)=N/C(=N\S(=O)(=O)c1ccc(C#N)cc1)NC12CC3CC(CC(C3)C1)C2 ; C/C(N)=N/C(=N\S(=O)(=O)c1ccc(OC(F)(F)F)cc1)NC12CC3CC(CC(C3)C1)C2] 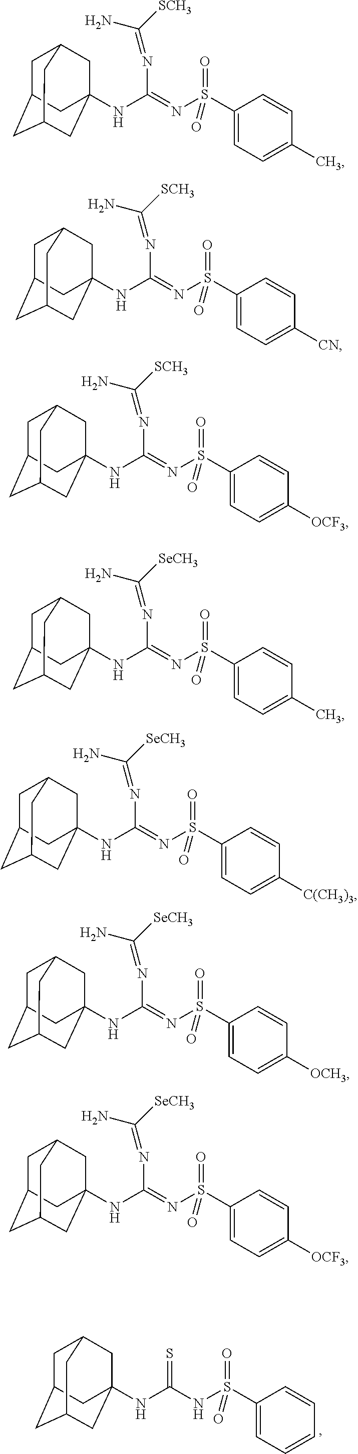 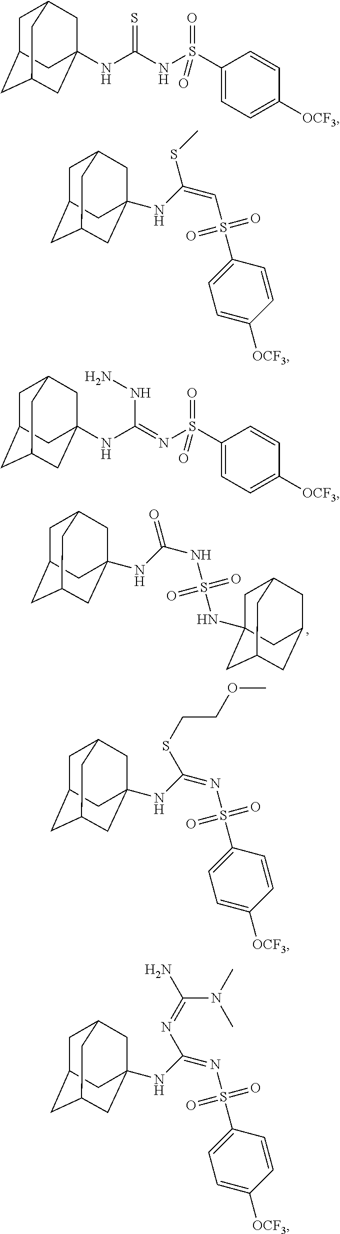 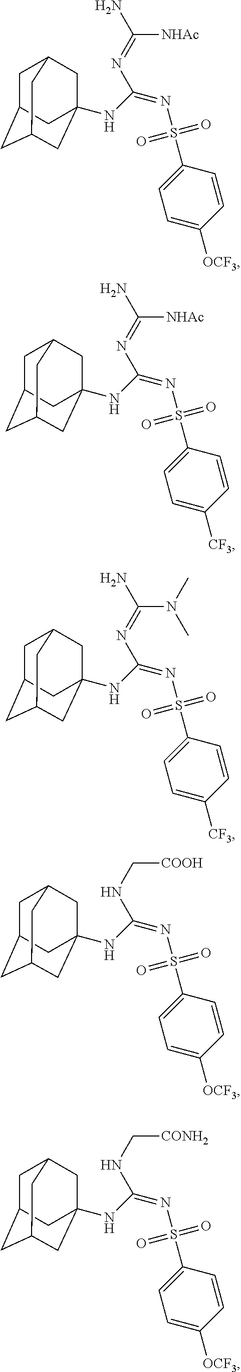 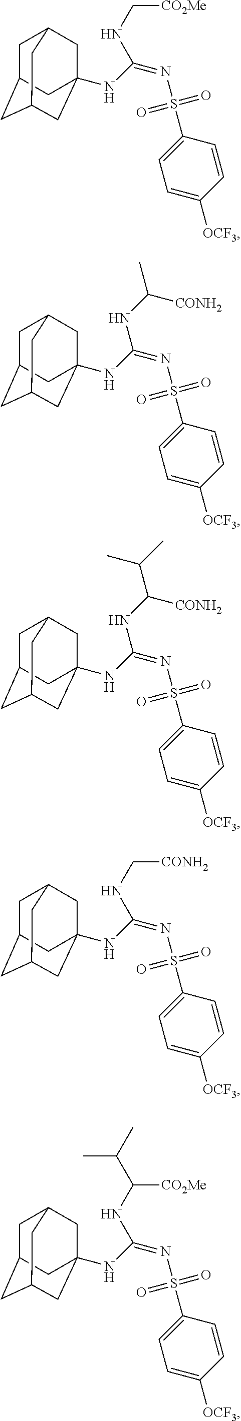 [SMILES: COC(=O)CN/C(=N/S(=O)(=O)c1ccc(OC(F)(F)F)cc1)NC12CC3CC(CC(C3)C1)C2 ; CC(N/C(=N/S(=O)(=O)c1ccc(OC(F)(F)F)cc1)NC12CC3CC(CC(C3)C1)C2)C(N)=O ; CC(C)C(N/C(=N/S(=O)(=O)c1ccc(OC(F)(F)F)cc1)NC12CC3CC(CC(C3)C1)C2)C(N)=O ; NC(=O)CN/C(=N/S(=O)(=O)c1ccc(OC(F)(F)F)cc1)NC12CC3CC(CC(C3)C1)C2 ; COC(=O)C(N/C(=N/S(=O)(=O)c1ccc(OC(F)(F)F)cc1)NC12CC3CC(CC(C3)C1)C2)C(C)C] 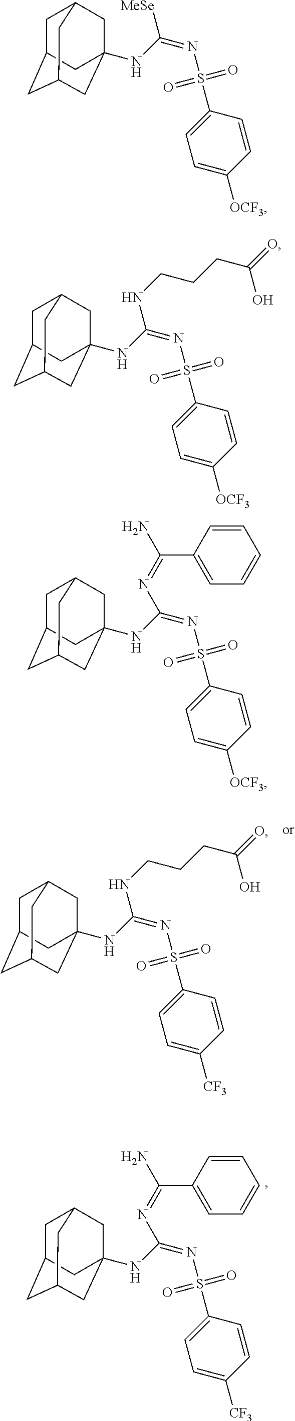
wherein -Me is methyl, and —NHAc is —NHC(═O)CH₃.

22. A pharmaceutical composition comprising a compound of claim 1, or a pharmaceutically acceptable ester, amide, solvate, salt, prodrug, or metabolite thereof, or a salt of such an ester, amide, prodrug, or metabolite, or a solvate of such an ester, amide, salt, prodrug, or metabolite together with a pharmaceutically acceptable carrier.

23. A method of treating an immunological disorder, or a condition treatable or preventable by inhibition of soluble epoxide hydrolase, in a patient, the method comprising providing to a patient in need thereof a compound of claim 1, or a pharmaceutically acceptable ester, amide, solvate, salt, prodrug, or metabolite thereof, or a salt of such an ester, amide, prodrug, or metabolite, or a solvate of such an ester, amide, salt, prodrug, or metabolite or a pharmaceutical composition of claim 22.

24. The method of claim 23, wherein said condition is hypertension, atherosclerosis, a pulmonary disease, diabetes, pain, fibrosis, addictive disorders, or inflammation.

25. The method of claim 23, further comprising administering to the patient in need thereof at least one additional therapeutic agent.
